SEQUENCE LISTING (20 sequences)

SEQ ID NO: 1 - DNA, 1257 bp, Mus musculus
atggctttcattgcagctctggggctcttgatggctgggatctgccctgctgtcctctgcttcccagatggcacgttgggaatggatgctgcagtccaagaagaccatgacaatgggacacaactggacagtctcacattggcctccatcaacactgactttgccttcagcctctacaaggagctggttttgaagaatccagataaaaatattgtcttctccccacttagcatctcagcggccttggctgtcatgtccctgggagcaaagggcaacaccctggaagagattctagaaggtctcaagttcaatcttacagagacctctgaggcagacatccaccagggctttgggcacctcctacagaggctcaaccagccaaaggaccaggtacagatcagcacgggtagtgccctgtttattgaaaagcgccagcagatcctgacagaattccaggagaaggcaaagactctgtaccaggctgaggccttcacagcagacttccagcagcctcgacaggccaaaaagctcatcaatgactatgtgaggaaacagacccaggggatgatcaaggaactggtctcagacctggataaaaggacattgatggtgctggtgaattatatctactttaaagccaaatggaaggtgccctttgaccctcttgacacgttcaagtctgagttctactgcggcaagaggaggcccgtgatagtgcccatgatgagcatggaggacctgaccacaccctacttccgagatgaggagctgtcctgcactgtggtggagctgaagtacacaggaaatgccagcgccctgttcatcctccctgaccagggcaggatgcagcaggtggaagccagcttacaaccagagaccctgaggaagtggaagaattctctgaaacccaggatgatagatgagctccacctgcccaagttctccatctccaccgactacagcctggaggatgtcctttcaaagctgggcatcagggaagtcttctccacacaggctgacctgtctgcaatcacaggaaccaaggatctgagagtgtctcaggtggtccacaaggctgtgctggatgtggctgagacaggcacagaagcagctgctgccactggagtcaaatttgtcccaatgtctgcgaaactgtaccctctgactgtatatttcaatcggcctttcctgataatgatctttgacacagaaactgaaattgccccctttatagccaagatagccaaccccaaatga

SEQ ID NO: 2 - protein, 418 aa, Mus musculus
MAFIAALGLLMAGICPAVLCFPDGTLGMDAAVQEDHDNGTQLDSLTLASINTDFAFSLYKELVLKNPDKNIVFSPLSISAALAVMSLGAKGNTLEEILEGLKFNLTETSEADIHQGFGHLLQRLNQPKDQVQISTGSALFIEKRQQILTEFQEKAKTLYQAEAFTADFQQPRQAKKLINDYVRKQTQGMIKELVSDLDKRTLMVLVNYIYFKAKWKVPFDPLDTFKSEFYCGKRRPVIVPMMSMEDLTTPYFRDEELSCTVVELKYTGNASALFILPDQGRMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 3 - protein, 247 aa, Homo sapiens
MQPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 4 - protein, 26 aa, Mus musculus
GTEAAAATGVKFVPMSAKLYPLTVYF

SEQ ID NO: 5 - protein, 8 aa, Mus musculus
KLINDYVR

SEQ ID NO: 6 - protein, 9 aa, Mus musculus
ELVSDLDKR

SEQ ID NO: 7 - protein, 10 aa, Mus musculus
VPFDPLDTFK

SEQ ID NO: 8 - protein, 10 aa, Mus musculus
QQILTEFQEK

SEQ ID NO: 9 - protein, 12 aa, Mus musculus
GNTLEEILEGLK

SEQ ID NO: 10 - protein, 14 aa, Mus musculus
MQQVEASLQPETLR

SEQ ID NO: 11 - protein, 16 aa, Mus musculus
EVFSTQADLSAITGTK

SEQ ID NO: 12 - protein, 19 aa, Mus musculus
AVLDVAETGTEAAAATGVK

SEQ ID NO: 13 - protein, 10 aa, Homo sapiens
VAQGIVSYGR

SEQ ID NO: 14 - protein, 9 aa, Homo sapiens
HSHTLQEVK

SEQ ID NO: 15 - protein, 12 aa, Homo sapiens
EQEPTQQFIPVK

SEQ ID NO: 16 - protein, 8 aa, Mus musculus
FVPMSAKL

SEQ ID NO: 17 - protein, 8 aa, Homo sapiens
VVAECCME

SEQ ID NO: 18 - protein, 8 aa, Homo sapiens
IIEFCCAS

SEQ ID NO: 19 - DNA, 31 bp, Artificial sequence
  primer
cgcggatccatggctttcattgcagctctgg

SEQ ID NO: 20 - DNA, 63 bp, Artificial sequence
  primer
cgcctcgagtcaggcgtagtcggggacgtcgtaggggtagaatttggggttcgctatcttggc

(19) United States
(12) Patent Application Publication
(10) Pub. No.: US 2007/0104699 A1
(43) Pub. Date: May 10, 2007

(54) Compositions for and methods of granzyme B inhibition

(75) Inventors: Ray V. Rajotte, Edmonton (CA); R. Chris Bleackley, Edmonton (CA); Greg Korbutt, Edmonton (CA); Sarah J. Lord, Edmonton (CA); Simonetta Sipione, Edmonton (CA); Katia Carmine-Simmen, Edmonton (CA); Fabrizio Giuliani, Edmonton (CA)

Correspondence Address: CLARK & ELBING LLP, 101 FEDERAL STREET, BOSTON, MA, 02110, US

(73) Assignee: University of Alberta, Edmonton (CA)

(21) Appl. No.: 11/527,373

(22) Filed: Sep. 26, 2006

Related U.S. Application Data
(60) Provisional application No. 60/721,799, filed on Sep. 29, 2005.

(51) Int. Cl. A61K 48/00; A61K 38/54; C12N 5/08

(52) U.S. Cl. 424/93.21; 435/325; 435/366; 424/94.2

(57) ABSTRACT

The present invention is related to the discovery that serpina3n, a secreted protein, binds to and inhibits granzyme B activity. The invention thus provides cells that include a polynucleotide encoding a granzyme B inhibitory serpin, pharmaceutical compositions including a granzyme B inhibitory serpin or a polynucleotide encoding a granzyme B inhibitory serpin, methods for treating a patient in need of immunosuppression by administration of a granzyme B inhibitory serpin, and methods of transplanting cells (e.g., islet cells) expressing a granzyme B inhibitory serpin.

### CROSS-REFERENCE TO RELATED APPLICATIONS

This application claims the benefit of U.S. Provisional Application No. 60/721,799, filed Sep. 29, 2005, which is hereby incorporated by reference.

### BACKGROUND OF THE INVENTION

Cytotoxic T lymphocytes (CTLs) provide essential protection against invading viruses and intracellular pathogens. There are however pathogenic contexts where these cells can cause harm to the body itself: cases include autoimmune disease (e.g., diabetes mellitus type 1, rheumatoid arthritis, Wegener's granulomatosis, and multiple sclerosis), graft (e.g., pancreatic islet cells) rejection, and graft-versus-host disease, inflammatory vascular disease, among others.

A major mechanism of CTL-mediated killing is the granzyme B pathway. When a CTL comes into contact with a target cell it delivers a “lethal hit” of cytolytic molecules that include perforin and granzyme B and result in death of the target cell by apoptosis. Briefly, the CTL-granzyme B pathway involves the calcium-dependent release of granzyme B and perforin, stored in the CTL lytic granules, in the direction of the target cell. Granzyme B, a mannose-6 phosphorylated (M6P) protein, binds its receptor, the mannose-6 phosphate/insulin-like growth factor-II (M6P/IGF-II) receptor, on the surface of the target cell and along with perforin is endocytosed by the target cell. Once inside the target cell, granzyme B remains in the endocytic vesicle, unable to mediate apoptosis, until released into the cytoplasm by perforin or another lytic agent (e.g., adenovirus). Once in the cytoplasm, granzyme B, a serine proteinase, cleaves pro-caspases at aspartic acid residues, activating them and initiating the caspase cascade to DNA fragmentation and apoptotic cell death.

Sertoli cells protect islets from auto-, allo-, and even xenoimmune mechanisms of graft destruction. Sertoli cell mediated protection of islets in the NOD mouse model, a model of autoimmune diabetes, has been attributed to TGF-β secreted by Sertoli cells. TGF-β is an anti-inflammatory cytokine capable of suppressing T-cell, macrophage, natural killer cell, and B-cell activity as well as the expression of many proinflammatory cytokines. Co-transplantation of islets with Sertoli cells isolated from rodent testis successfully protects islets from allo- and autoimmune mechanisms of graft destruction. However, prior to the present invention, how Sertoli cells achieve this feat was poorly understood.

It is therefore critical to find methods for inhibiting CTL activity for successful treatment of pathogenic conditions involving these cells. Such methods can be used in the treatment of autoimmune disorders (e.g., diabetes or rheumatoid arthritis), an inflammatory vascular disease, or an inflammatory neuronal disease and can protect transplanted tissue from rejection.

### SUMMARY OF THE INVENTION

Based on our identification of serpina3n as a secreted granzyme B inhibitory serpin, the present invention provides methods for treatment of patients in need of immunosuppression, compositions useful in the treatment of such patients, and methods for transplantation of cells into a patient. Accordingly, in a first aspect the present invention provides a method for treating a patient in need of immunosuppression (e.g., a patient with an autoimmune disorder such as diabetes, rheumatoid arthritis, or any autoimmune disorder listed herein, an inflammatory vascular disease, or an inflammatory neuronal disease or a patient that has received a transplanted cell, which may be part of a transplanted organ, for example, a heart, liver, kidney, pancreas, or lung). The method includes administering to the patient a therapeutically effective amount of a composition including a granzyme B inhibitory serpin (e.g., serpina3n or modified human α1-antichymotrypsin) or a granzyme B inhibitory fragment thereof in an amount sufficient to decrease an immune response (e.g., an immune response mediated by cytotoxic T lymphocytes) of the patient. The granzyme B inhibitory serpin may be a secreted protein.

In a second aspect, the invention provides a method for transplanting a cell into a mammal (e.g., a human) which includes providing a composition including a first cell including a first heterologous polynucleotide encoding a granzyme B inhibitory serpin (e.g., serpina3n or modified human α1-antichymotrypsin) or a granzyme B inhibitory fragment thereof, where the cell (e.g., an islet cell, a human cell, a stem cell, a porcine cell, or a fish cell such as a Brockmann body) is a eukaryotic cell; and introducing the composition into the mammal. The composition may further include a second cell (e.g., an islet cell). The cell may be a cell in a transplanted organ (e.g., a heart, liver, kidney, pancreas, or lung). The cell may further include a second heterologous polynucleotide encoding a second polypeptide (e.g., insulin such as human insulin).

In a third aspect, the invention provides a composition including a cell (e.g., a mammalian cell such as a human cell, a porcine cell, an islet cell, a stem cell, a fish cell such as a Brockmann body) including a heterologous polynucleotide sequence encoding a granzyme B inhibitory serpin (e.g., serpina3n or modified human α1-antichymotrypsin) or a granzyme B inhibitory fragment thereof, where the cell is a eukaryotic cell. The polynucleotide sequence may be operably linked to a promoter. The composition may further include a second cell for transplantation (e.g., an islet cell).

In a fourth aspect, the invention provides a pharmaceutical composition including a granzyme B inhibitory serpin (e.g., serpina3n or modified human α1-antichymotrypsin) or a granzyme B inhibitory fragment thereof and a pharmaceutically acceptable carrier (e.g., suitable for parenteral administration or intravenous administration).

In a fifth aspect, the invention provides a pharmaceutical composition including a polynucleotide encoding a granzyme B inhibitory serpin (e.g., serpina3n or modified human α1-antichymotrypsin) or a granzyme B inhibitory fragment thereof and a pharmaceutically acceptable carrier.

In a sixth aspect, the invention provides a composition including a vector (e.g., a viral vector) including a polynucleotide encoding a granzyme B inhibitory serpin or a granzyme B inhibitory fragment thereof.

In a seventh aspect, the invention provides a transgenic, non-human animal (e.g., a pig or a fish) including a first heterologous polynucleotide encoding a granzyme B inhibitory serpin or granzyme B inhibitory fragment thereof, wherein the serpin or the fragment is operably linked to a promoter capable of expressing the polynucleotide in at least one tissue (e.g., cardiac or pancreatic tissue) of the transgenic animal. The transgenic animal may further include a second heterologous polynucleotide (e.g., a polynucleotide encodes human insulin).

In an eighth aspect, the invention provides a method for transplanting tissue from a transgenic animal (e.g., a pig or fish) into a patient (e.g., a human). The method includes providing a composition including a tissue (e.g., cardiac or pancreatic tissue, tissue including a heart, liver, kidney, pancreas, or lung, tissue including an islet cell) from the transgenic animal of the seventh aspect and introducing the composition into the patient.

By a “granzyme B inhibitory serpin” is meant a polypeptide with at least 30%, 40%, 50%, 60%, 70%, 80%, 90%, 95%, or 99% sequence identity to serpina3n (SEQ ID NO:2; see FIG. 8) or a polypeptide encoded by a polynucleotide that hybridizes (e.g., under stringent conditions) to the polynucleotide encoding serpina3n (SEQ ID NO:1; see FIG. 8), where the polypeptide inhibits mammalian granzyme B activity (e.g., human granzyme B (SEQ ID NO: 3; see FIG. 8)). In addition, the term granzyme B inhibitory serpin encompasses any other serpin protein modified to inhibit granzyme B (e.g., by specifically binding granzyme B). Modifications may include substitution of a reactive center loop (RCL) for an heterologous RCL (e.g., the RCL of serpina3n) that confers granzyme B inhibitory (e.g., binding) activity to the serpin. In one embodiment, human α1-antichymotrypsin is modified to contain the RCL of mouse serpina3n. Specifically excluded from this definition are SPI-6 and PI-9 and sequences with 85%, 90%, 95%, 98%, 99%, or greater homology to SPI-6 or PI-9. Granzyme B inhibitory serpins may include homologues and xenologues from any organism, for example, from a mammal such as a rat, a pig, a human, or a mouse, and may include a serpin with sequence derived from such homologues and xenologues. In any aspect of the invention, the granzyme B inhibitory serpin can be a secreted protein (e.g., containing a sequence that targets the polypeptide for secretion) when produced by a cell (e.g., a mammalian cell).

By “granzyme B inhibitory serpin fragment” is meant a fragment of at least four amino acids of a granzyme B inhibitory serpin that retains at least 1%, and preferably 5%, 10%, 25%, 50%, 75%, 90%, 95%, 99%, or even 100% of the granzyme B inhibitory activity of the full length granzyme B inhibitory serpin from which it is derived. Granzyme B inhibitory activity may be measured as described herein. In certain embodiments, a granzyme B inhibitory serpin fragment contains a granzyme B inhibitory RCL.

By “serpin” is meant a serine protease inhibitor. Serpins include the mouse α1-antitrypsin (or α1-protease inhibitor) family, human serpins such as α1-antitrypsin and α1-antichymotrypsin, and homologues or xenologues of such proteins. Serpins are found, for example, in organisms including rat, pig, yeast, and C. elegans. Serpins may have a reactive center loop (RCL) through which specificity to a target serine protease may be mediated.

By “fragment” is meant a portion of polypeptide that is at least 4 amino acids and retains at least a fraction of the biological activity (e.g., granzyme B binding) of the full length polypeptide. Preferably, a fragment retains at least 1%, 5%, 10%, 25%, 50%, 75%, 90%, 95%, or 99% of the activity of the full length polypeptide.

By “modified” is meant any change to a molecule (e.g., a polypeptide). Modifications of, for example, polypeptides include a mutation such as an insertion, deletion, or amino acid substitution, or may include modifications to side chain amino acid residues such as methylation, or oxidation.

By “granzyme B inhibitory reactive center loop” or “granzyme B inhibitory RCL” is meant a region of a serpin that includes a short (e.g., 19 amino acids) stretch of amino acids that confers specificity to granzyme B of a serpin. An exemplary granzyme B inhibitory RCL (GTEAAAATGVKFVPMSAKLYPLTVYF (SEQ ID NO:4)) is contained within the serpina3n sequence. A covalent linkage between the granzyme B inhibitory RCL and granzyme B may form following cleavage of the RCL by granzyme B, resulting in irreversible inactivation of granzyme B. Granzyme B inhibitory activity of a specific RCL may be determined using the methods described herein (e.g., by mixing granzyme B with IEDP-pNA and comparing cleavage of the IEDP-pNA by granzyme B in the presence and in the absence of a polypeptide containing a granzyme B inhibitory RCL). Specific residues important in granzyme B inhibitory activity may be identified using mutagenic techniques standard in the art, for example, as described by Sun et al. (J. Biol. Chem. 276:15177-15184 (2001)), and such methods may be used to identify a novel granzyme B inhibitory RCL.

By “granzyme B inhibition” is meant a reduction of granzyme B activity of at least 5%, and preferably 10%, 25%, 50%, 75%, 90%, 95%, 99%, or even 100%. Granzyme B activity may be measured using any number of methods known in the art. One such method includes mixing granzyme B with isoleucine/glutamate/proline/aspartate conjugated to paranitroanalide (IEPD-pNA), which contains a cleavage site for granzyme B. Cleavage of IEPD-pNA by granzyme B produces IEPD and pNA, a colored product, whose absorbance can be measured at 405 nm and is proportional to the amount of granzyme B enzymatic activity in the assay. A molecule (e.g., a polypeptide such as a serpin) may inhibit granzyme B by specifically binding to the active site granzyme B. Measurements of granzyme B activity can also be performed using a cell killing assay (e.g., those described herein).

By “specifically binds” is meant a compound (e.g., a first polypeptide) or antibody which recognizes and binds another molecule (e.g., a second polypeptide) but which does not substantially recognize and bind other molecules in a sample, for example, a biological sample, which naturally includes a polypeptide.

By “promoter” is meant a minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “pharmaceutically acceptable carrier” means a carrier which is physiologically acceptable to the treated mammal while retaining the therapeutic properties of the compound or cells with which it is administered. One exemplary pharmaceutically acceptable carrier is physiological saline. Other physiologically acceptable carriers and their formulations are known to one skilled in the art and described herein and, for example, in Remington's Pharmaceutical Sciences, (18^{th} edition), ed. A. Gennaro, 1990, Mack Publishing Company, Easton, Pa.

By “treating” is meant administering of a pharmaceutical composition for the treatment or prevention of a disease or of a symptom associated with a disease.

By “CTL-mediated disease” is meant a disease in which CTL cells inappropriately target a cell for death. A CTL-mediated disease may be an autoimmune disorder (e.g., diabetes), an inflammatory vascular disease, an inflammatory neuronal disease, or a transplant situation.

By “cell for transplantation” is meant any cell which may be provided to a patient (e.g., a human). Cells suitable for transplantation may include cells from the patient, cells taken from another animal (e.g., a cell taken from an animal of the same species or a different species), or cells taken from a cadaveric donor. Particularly useful cells in the present invention include pancreatic islet cells, and particularly useful sources of these cells include fish, pigs, and human.

By “autoimmune disorder” refers to a disorder wherein the immune system of a mammal mounts a humoral or cellular immune response to the mammal's own tissue or has intrinsic abnormalities in its tissues preventing proper cell survival without inflammation.

Examples of autoimmune diseases include, but are not limited to, diabetes, rheumatoid arthritis, inflammatory neurodegenerative disease (e.g., multiple sclerosis), lupus erythematosis, myasthenia gravis, scieroderma, Crohn's disease, ulcerative colitis, Hashimoto's disease, Graves' disease, Sjogren's syndrome, polyendocrine failure, vitiligo, peripheral neuropathy, graft-versus-host disease, autoimmnune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barr syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, and thyroiditis.

By “inflammatory vascular disease” is meant any condition associated with inflammation of vascular tissue. Such diseases may be mediated by increased endothelial cell apoptosis or the granzyme B apoptotic pathway. Exemplary inflammatory vascular diseases include atherosclerosis, meningitis, temporal arteritis, transplant vascular disease, Takayasu arteritis, giant cell arteritis, aortic aneurysm, meningitis, and temporal arteritis.

By “inflammatory neuronal disease” is meant any condition associated with inflammation of nervous tissue (e.g., neurons). In certain cases, such diseases may be mediated by the granzyme B apoptotic pathway. Inflammatory neuronal diseases include multiple sclerosis, Parkinson's disease, amyotrophic lateral sclerosis (ALS), Huntington's disease, prion disease (e.g., Creutzfeldt-Jakob disease and scrapie), and Alzheimer's disease.

By “sufficient to decrease an immune response in a patient” is an amount of a composition (e.g., a composition with immunosuppressive activity), upon administration to a patient, having the ability to reduce at least one immune response (e.g., CTL-mediated killing) by 5%, 10%, 25%, 50%, 75%, 90%, 95%, 97%, 98%, 99%, or more.

By “immunosuppressive activity” is meant a reduction of at least one immune response (e.g., CTL-mediated killing). The reduction may be at least 2%, 5%, 10%, 25%, 50%, 75%, 90%, 95%, 97%, 98%, 99%, or more.

Other features and advantages of the invention will be apparent from the following Detailed Description, the drawings, and the claims.

### BRIEF DESCRIPTION OF THE DRAWINGS

FIGS. 1A and 1B are graphs showing that Sertoli cell-conditioned media (SCCM) reduces granzyme B-mediated killing. FIG. 1A shows ³H-thymidine release from L-cells after 3 hour incubation with a CTL line in the presence of HAM's control media or SCCM. FIG. 1B shows TUNEL labeling of L-cells after a three hour incubation with 24, 120, or 600 ng/ml granzyme B and adenovirus in the presence of HAM's control media or SCCM. Data shown as the mean±SEM of at least three different experiments carried out on different preparations of SCCM. A star (*) indicates a significant reduction (p<0.05) in killing upon treatment with SCCM.

FIGS. 2A-2D are graphs showing SCCM has no effect on mannose-6 phosphate receptor (MPR) expression or granzyme B (grB) uptake. FIGS. 2A and 2B show cation independent (CI)- and cation dependent (CD)- forms of the MPR expression in L-cells after 1 hour incubation in the presence of HAM's control media or SCCM. MPR expression was determined using antibodies specific for CI- and CD-MPR followed by incubation with a FITC conjugated secondary antibody and flow cytometric analysis. FIGS. 2C and 2D show binding and uptake of granzyme B in L-cells after a one hour incubation in the presence of HAM's control media or SCCM. Granzyme B was conjugated to Alexa 488 for the determination of binding and uptake in L-cells through flow cytometric analysis. Data are presented as the relative mean fluorescence intensity (MFI) (FIGS. 2B and 2D) or percent positive cells (FIGS. 2A and 2C). Data are shown as the mean±SEM of at least three independent experiments carried out on different preparations of SCCM.

FIGS. 3A and 3B are graphs showing that SCCM reduces granzyme B enzymatic activity. FIG. 3A shows cleavage of IEPD-pNA by human purified granzyme B at three different concentrations of granzyme B (24, 120, or 600 ng/ml) in the presence of HAM's control media or SCCM. FIG. 3B shows cleavage of IEPD-pNA by mouse CTL degranulate granzyme B in the presence of HAM's control media or SCCM. Cleavage of IEPD-pNA by granzyme B results in the release of pNA whose absorbance is measured at 405 nm. Data shown as the mean±SEM of at least three different experiments carried out on different preparations of SCCM. A star (*) indicates a significant reduction (p<0.05) in activity upon treatment with SCCM.

FIGS. 4A-4C are images of Western blots showing that granzyme B is covalently modified by a factor (i) secreted by cultured Sertoli cells and (ii) is not SPI-6. FIGS. 4A-4C show Western blots of granzyme B incubated for two hours with HAM's control media, SCCM, or PBS. FIG. 4A shows detection with an anti-granzyme B antibody. Each lane is as follows. 1) HAM's, 2) SCCM, 3) HAM's+granzyme B, 4) SCCM+granzyme B, 5) granzyme B. The arrow indicates a higher molecular mass band that appears in lane 4 with SCCM and granzyme B. FIG. 4B shows a Western blot using an anti-SPI-6 antibody. FIG. 4C shows the same blot as in FIG. 4B stripped and re-probed with an anti-granzyme B antibody. Each lane of FIGS. 4B and 4C is as follows. 1) HAM's, 2) SCCM, 3) HAM's+granzyme B, 4) SCCM+granzyme B, 5) granzyme B, 6) mouse CTL lysate. The arrow indicates the higher molecular mass complex that appears when granzyme B is incubated with SCCM and that is not detected by the SPI-6 antibody.

FIGS. 5A and 5B are images of Western blots showing that serpina3n forms a complex with granzyme B in vitro. FIG. 5A shows SDS-PAGE and autoradiography of in vitro translated/transcribed and ³⁵S-radiolabeled serpina3n-HA incubated with human granzyme B (300 ng) or PBS. Each lane is as follows. 1) ³⁵S-serpina3n-HA+PBS, 2) ³⁵S-serpina3n-HA+grB, 3) reticulocyte lysate+grB. FIG. 5B shows granzyme B immunoblot after incubation of in vitro translated/transcribed serpina3n-HA with human granzyme B (85 ng) or PBS. Each lane is as follows. 1) Serpina3n-HA+PBS, 2) Serpina3n-HA+grB, 3) reticulocyte lysate+grB, 4) reticulocyte lysate. Data shown are representative of three independent experiments.

FIGS. 6A and 6B are images of Western blots showing that transfected Jurkat cells secrete serpina3n, which binds to granzyme B. FIG. 6A shows expression of serpina3n-HA in Jurkat cells. 5×10⁶ stable transfected cells (clone SerE12-HA) were incubated overnight in 1 ml OPTI-MEM I medium. Serpina3n in the cell lysate (L) and in the conditioned medium (CM) was detected by immunoblotting with anti-HA antibody. FIG. 6B shows serpina3n-HA secreted into the culture medium formed a complex with human granzyme B. Purified human granzyme B was incubated for two hours at 37° C. with medium collected from Jurkat cells, pcDNA3-transfected cells or SerE12-HA cells. Formation of serpina3n-granzyme B complex was detected by SDS-PAGE and immunoblotting with an anti-granzyme B antibody.

FIG. 6C is a graph showing serpina3n-HA inhibits granzyme B enzymatic activity. Granzyme B (212 ng) was pre-incubated for 1 hr at 37° C. with increasing volumes of conditioned medium from SerE12-HA cells or pcDNA3-transfected cells, and then granzyme B activity was measured. Data are expressed as percentage of the activity of granzyme B pre-incubated with the medium of pcDNA3-transfected cells and are the mean±standard deviation of three independent experiments performed in triplicate.

FIG. 7 is a graph showing quantitative analysis of neuron survival following exposure to anti-CD3 activated T-cells or recombinant Granzyme B. The number of MAP-2 positive neurons remaining after co-culture with activated T-cells alone or pre-treated for two hours with different conditions (concentrated (5×)AIMV, concentrated Jurkat cell supernatant, concentrated F8 supernatant, or concentrated Jurkat cells supernatant containing serpina3n) is shown. Almost 60% of neurons are lost in the co-culture with recombinant granzyme B or activated T-cells alone. Only 30% of neurons were lost in the co-culture with activated T-cells pre-treated with serpina3n. *p<0.01 one-way ANOVA with Tukey's post-hoc test. Unact=Unactivated T-cells; ACT=activated T-cells; p.t.=pre-treatment.

FIG. 8 shows the polynucleotide (SEQ ID NO:1) and polypeptide (SEQ ID NO:2) sequences of serpina3n, the polypeptide sequence of granzyme B (SEQ ID NO:3), and the polypeptide sequence of the serpina3n reactive center loop (SEQ ID NO:4).

### DETAILED DESCRIPTION

The present invention features compositions and methods for the treatment of patients in need of immunosuppression such as those patients having an autoimmune disorder (e.g., diabetes, rheumatoid arthritis), an inflammatory vascular disease, or transplant. Compositions include cells containing polynucleotides encoding serpina3n and pharmaceutical compositions useful in the treatment of patients in need of immunosuppressive therapy.

In the present study, we identified a novel activity for a factor, serpina3n, secreted by Sertoli cells that inhibits CTL killing via blocking the granzyme B pathway to apoptotic target cell death, a main immune effector mechanism in graft destruction. One possibility was that Sertoli cells inhibited granzyme B-mediated apoptosis through the secretion of ligands for the M6P/IGF-II receptor. However, the Sertoli cell-conditioned medium (SCCM) had no effect on M6P/IGF-II receptor cell-surface expression, nor did SCCM interfere with granzyme B binding or uptake, raising the possibility that inhibitory action of the SCCM results from a direct effect on granzyme B proteolytic activity. As shown here, a factor, serpina3n, secreted by mouse Sertoli cells effectively reduced both human and mouse granzyme B enzymatic activity by a direct interaction with granzyme B.

As detailed below, when human granzyme B was incubated in mouse SCCM, a stable complex containing granzyme B forms. The granzyme B complex was resistant to SDS and heat-induced denaturation, consistent with the complex including a serine proteinase inhibitor (serpin). The complex with granzyme B did not include SPI-6, which indicated that another serpin secreted by Sertoli cells must be interacting with granzyme B to form an SDS-stable complex. Indeed, MALDI-TOF mass spectrometry analysis of the complex unequivocally identified a different serpin, serpina3n, as the factor bound to granzyme B. Cloning and expression of serpina3n in Jurkat cells confirmed that this protein binds to and inhibits granzyme B activity. This is the first observation of a serpin other than PI-9 or SPI-6 inhibiting granzyme B. Further, serpina3n, unlike PI-9 or SPI-6, is a secreted protein.

The observation of a novel granzyme B inhibitor secreted by Sertoli cells contributes to understanding of the mechanism by which Sertoli cells protect islet grafts from allo-, auto-, and xeno-immune mechanisms of destruction. Secreted serpina3n effectively inhibits granzyme B activity and granzyme B-mediated killing and this mechanism represents a powerful and novel approach to blocking the host cell-mediated immune response. The present invention therefore provides methods of allo- and xeno-transplantation and co-transplantation, as well as other forms of immunnosuppression, by providing a serpin.

Granzyme B

Granzyme B is an important member of the granzyme family. Granzyme B and perforin are the effector molecules that mediate target killing by NK cells and CTLs in viral infection and anti-tumor immunity. Perforin is normally required for granzyme B activity as perforin mediates cell entry of granzyme B; however, there are a number of cases where the granzyme B substrate is on the outside of a cell, and in these cases, perforin is not required (Choy et al., Arterioscler. Thromb. Vasc. Biol. 24:2245-2250 (2004)). Dysregulation of this pathway is associated with certain human diseases and genetic abnormalities in mice (Russell et al., Annu. Rev. Immunol. 20:323-370 (2002)). Granzyme B and perforin work synergistically to exert a cytotoxic effect on target cells. The mechanisms underlying the delivery of granzyme B to target cells may involve transmembrane pores made by perforin (Yagita et al., Adv. Immunol. 51:215-242 (1992)), nonspecific charge interaction (Shi et al., J. Immunol. 174:5456-5461 (2005)), and/or cation-independent mannose 6-P receptor-mediated endocytosis (Motyka et al., Cell 103:491-500 (2000)). Endothelial cell apoptosis is mediated by CTL cells. Granzyme B has been implicated in this process and thus may be involved in autoimmune diseases, inflammatory vascular diseases such as atherosclerosis, Takayasu arteritis, giant cell arteritis, inflammatory neuronal diseases, and diseases associated with organ transplantation such as transplant vascular disease (Choy et al., Arterioscler. Thromb. Vasc. Biol. 24:2245-2250 (2004); Choy et al., Am. J. Transplant. 5:494-499 (2005)). In addition, regulatory T cells use granzyme B to inhibit responses to tumors.

Serpin Family of Proteins

Serpina3n is a member of a multigene family of serpins with high degree of homology with the human α1-antichymotrypsin (SERPINA3). While in humans there is a single gene coding for α1-antichymotrypsin, repeated duplication events resulted in the appearance of a cluster of 14 closely related genes in mice (Forsyth et al., Genomics 81:336-345 (2003)). Among these genes, serpina3n is the one with the highest degree of homology with antichymotrypsin (61% at the amino acid level), at least for what concerns the structural part of the protein. Based on the amino acid sequence of its reactive center loop, it was proposed that serpina3n may function as an elastase (Horvath et al., J. Mol. Evol. 59:488-497 (2004)). More recent work has shown that serpina3n shares substrate specificity with both human antichymotrypsin and human antitrypsin and can bind and inactivate chymotrypsin, trypsin, cathepsin G, and elastase (Horvath et al., J. Biol. Chem. 280:43168-43178 (2005)). Here, we show that serpina3n is also an inhibitor of granzyme B.

The previously characterized inhibitors of granzyme B, PI-9 and SPI-6, require an acidic residue in P1 position of the reactive center loop to block granzyme B activity (Sun et al., J. Biol. Chem. 276:15177-15184 (2001); Sun et al., J. Biol. Chem. 272:15434-15441 (1997)). Other residues in the reactive center loop, specifically the residues P4-P4′, are important for the interaction with granzyme B (Sun et al., J. Biol. Chem. 276:15177-15184 (2001)). Although the reactive center loop of serpina3n does not contain acidic residues, it presents a Met in position P1 which can be cleaved by granzyme B (Poe et al., J. Biol. Chem. 266:98-103 (1991); Odake et al., Biochemistry 30:2217-2227 (1991)). Moreover, many of the residues P4-P4′ in the RCL of serpina3n are compatible with granzyme B specificity as defined by scanning mutagenesis of the PI-9 reactive center loop (Sun et al., J. Biol. Chem. 276:15177-15184 (2001)).

Serpina3n is highly expressed in brain, testis, lung, thymus, and spleen (Horvath et al., J. Mol. Evol. 59:488-497 (2004)). In testis, serpina3n secreted by Sertoli cells may act in concert with SPI-6 to modulate the activity of the locally produced granzyme B (Hirst et al., Mol. Hum. Reprod. 7:1133-1142 (2001)). A key difference between PI-9/SPI-6 and serpina3n is that the latter is a secreted polypeptide, whereas PI-9 and SPI-6 are intracellular.

Detailed below are the results of experiments demonstrating the inhibition of CTL-mediated cell death by SCCM containing serpina3n.

Sertoli Cells Protect Transplanted Islet Cells from CTL-mediated Apoptotic Death

Co-transplantation of islets with Sertoli cells isolated from rodent testis protects islets from xeno, allo-, and autoimmune mechanisms of graft destruction (Selawry et al., Cell Transplant. 2:123-129 (1993); Korbutt et al., Diabetes 46:317-322 (1997); Takeda et al., Diabetologia 41:315-321 (1998); Korbutt et al., Diabetologia 43:474-480 (2000)). Prior to the present invention, the mechanism by which Sertoli cells protect an islet cell was poorly understood. Sertoli cells, at least in part through the inhibition of CTL killing, are able to protect islet cells, and indeed, Sertoli cells have been found to express proteins which block the CTL-granzyme B pathway, thereby preventing apoptotic cell death. For example, Sertoli cells secrete M6P-glycoproteins and IGF-II, which are ligands for the M6P/IGF-II death receptor for granzyme B (O'Brien et al., Biol. Reprod. 49:1055-1065 (1993); Tsuruta et al., Biol. Reprod. 63:1006-1013 (2000)). M6P-glycoproteins expressed in Sertoli cells include prosaposin, procathepsin L, and transforming growth factor-beta (TGF-β) (O'Brien et al., Biol. reprod. 49:1055-1065 (1993); Russell et al., The Sertoli Cell, Clearwater, Fla.: Cache River Press (1993)). TGF-β, in particular, is an immunosuppressant agent implicated in Sertoli cell-mediated protection of islets in the NOD mouse (Suarez-Pinzon et al., Diabetes 49:1810-1818 (2000)). These proteins may downregulate or block the receptor, thereby preventing granzyme B uptake and subsequent target cell killing.

As described herein, the effect of SCCM on granzyme B mediated apoptosis was tested, and Sertoli cells were found to secrete a factor that inhibits granzyme B enzymatic activity through the formation of a stable complex which reduces granzyme B mediated apoptosis. This factor exhibited the characteristics of a serpin but was not murine serine proteinase inhibitor-6 (SPI-6), a murine inhibitor of granzyme B. Mass spectrometry analysis identified this factor as a new and novel inhibitor of granzyme B, serpina3n.

Sertoli Cell-Conditioned Media Affects Granzyme B-Mediated Killing

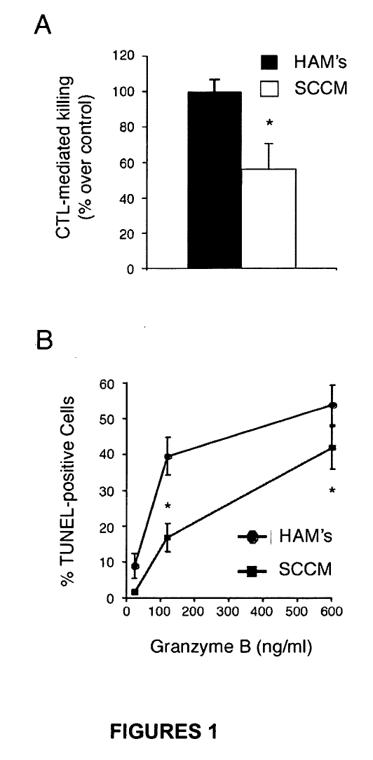

We first tested whether SCCM was able to protect target cells from CTL-mediated killing. ³H-thymidine labeled L1210 cells underwent apoptotic cell death (% specific ³H-thymidine release) when treated with a C57 CTL cell line. Killing by the C57 CTL line is primarily a result of granzyme B rather than a Fas ligand (data not shown). Treatment of L-cells with SCCM significantly reduced CTL killing (FIG. 1A).

To assess whether SCCM affected the granzyme B-mediated pathway of killing, a killing assay using purified granzyme B was performed. Granzyme B treatment of target cells resulted in dose-dependent DNA fragmentation and cell death, as assessed by TUNEL analysis. However, in the presence of SCCM, granzyme B-mediated DNA fragmentation in target cells was dramatically reduced (FIG. 1B). The reduction in DNA fragmentation was found to be significant at doses equal or higher than 120 ng/ml of granzyme B (p<0.05).

Sertoli Cell-Conditioned Media Inhibits Granzyme B Enzymatic Activity

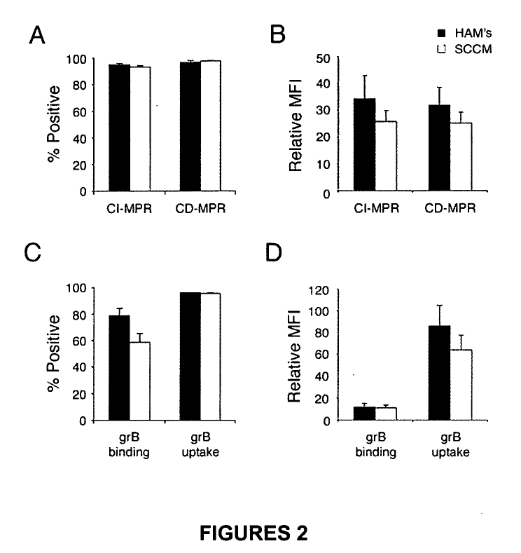

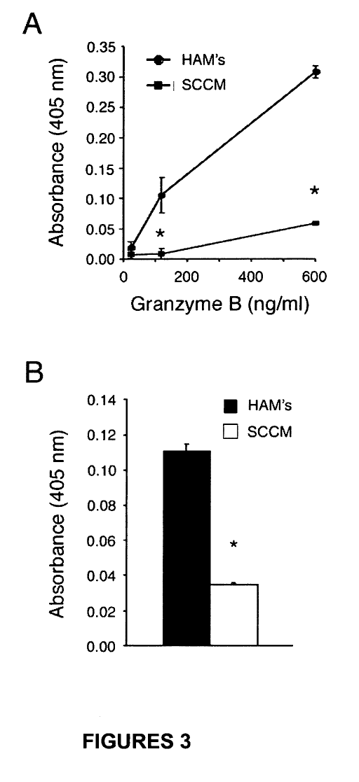

We found no significant effect of SCCM on M6P/IGF-II receptor expression or granzyme B uptake (FIGS. 2A-2D). Thus, it was determined whether the observed inhibition of target cell killing resulted from SCCM affecting granzyme B proteolytic activity. Indeed, pre-incubation of human granzyme B with SCCM resulted in significant reduction (83% decrease) of granzyme B activity, while no inhibition was observed when granzyme B was incubated with control HAM's F10 medium (FIG. 3A). Similar results were observed with mouse granzyme B obtained from CTL degranulate material (FIG. 3B).

Granzyme B is Covalently Modified by a Factor Secreted by Sertoli Cells

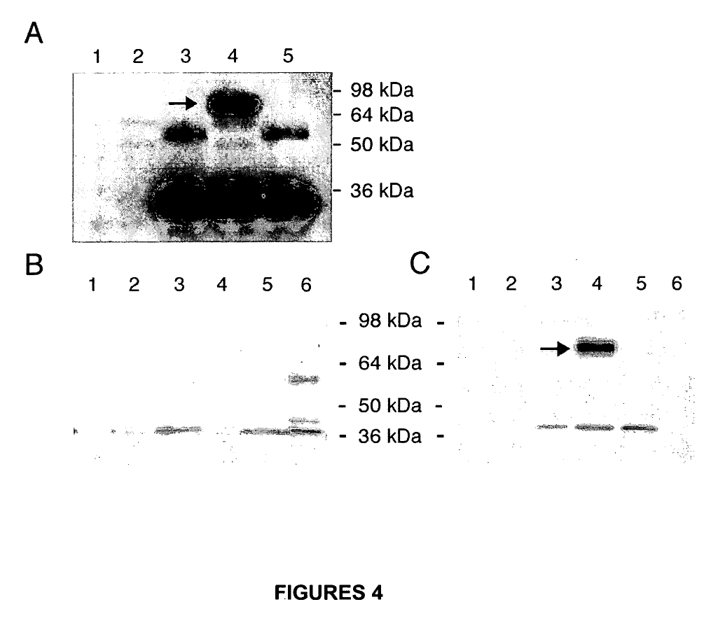

In order to assess whether granzyme B was modified by factors secreted from Sertoli cells, granzyme B was incubated with SCCM, which was then resolved by SDS-PAGE and Western blotting with an anti-granzyme B antibody. As shown in FIG. 4A, control samples (granzyme B alone and granzyme B incubated with HAM's F10 control medium) exhibit a band with an approximate molecular mass of 32 kDa, and a second band with a molecular mass of approximately 54 kDa was also observed, likely corresponding to a glycosylated form of granzyme B. When granzyme B was pre-incubated with SCCM, a new immunoreactive band appeared with an approximate molecular mass of 78 kDa, thus indicating a stable complex between granzyme B and a previously unknown factor in SCCM forms. We suspected this factor to be a serine proteinase inhibitor or serpin. Serpins are known to bind essentially irreversibly to their cognate proteinase in a manner resistant to SDS and heat-denaturation, a property thought to be unique among this class of proteinase inhibitors (Potempa et al., J. Biol. Chem. 269:15957-15960 (1994)). Prior to the present invention, the serine proteinase inhibitors known to inhibit granzyme B enzymatic activity through the formation of a stable complex were murine SPI-6 and human PI-9. Sertoli cells have been shown to express SPI-6 and PI-9 in mouse and human testis, respectively (Bladergroen et al., J. Immunol. 3218-3225 (2001); Hirst et al., Mol. Hum. Reprod. 7:1133-1142 (2001)). To determine whether SPI-6 is responsible for the binding and inhibition of granzyme B in SCCM, Western blotting with an antibody recognizing SPI-6 was performed. This experiment showed that no SPI-6 was detectable in SCCM or in the complex with granzyme B. An immunoreactive band with a molecular mass of 42 kDa was present in the positive control (total cell lysate from C57 mouse CTL) (FIG. 4B). FIG. 4C shows the position of the granzyme B complex in the same gel, stripped and reprobed with the anti-granzyme B antibody. These data indicate that SPI-6 is not observed in the complex that forms with granzyme B in SCCM.

Identification of a New Granzyme B Inhibitor Secreted by Mouse Sertoli Cells

In order to characterize the complex formed upon incubation of purified granzyme B with the Sertoli cell-conditioned medium, MALDI-TOF mass spectrometry analysis of the high-molecular mass complex immunoprecipitated with an anti-granzyme B antibody was performed. Two proteins were identified in the complex based on their peptide mass fingerprints (Table 1): human granzyme B and mouse serpina3n (also known as spi2.2), a serine proteinase inhibitor. The predicted molecular masses of serpina3n (47 kDa) and human granzyme B (32 kDa) are indeed compatible with the observed covalent heterodimeric complex with an apparent molecular mass of about 78 kDa.

TABLE 1

| | | | | |
|---|---|---|---|---|
| | | Mr | Mr | |
| Peptide | (Observed) | (Expected) | Delta | Ion Score |

| | | | | | |
|---|---|---|---|---|---|
| KLINDYVR | (SEQ ID NO:5) | 1019.58 | 1019.58 | 0.01 | 26 |
| ELVSDLDKR | (SEQ ID NO:6) | 1073.59 | 1073.57 | 0.02 | 40 |
| VPFDPLDTFK | (SEQ ID NO:7) | 1177.61 | 1177.60 | 0.01 | 27 |
| QQILTEFQEK | (SEQ ID NO:8) | 1262.66 | 1262.65 | 0.01 | 46 |
| GNTLEEILEGLK | (SEQ ID NO:9) | 1314.7 | 1314.7 | −0.00 | 59 |
| MQQVEASLQPETLR + oxidation (M) | (SEQ ID NO: 10) | 1644.85 | 1644.81 | 0.03 | 83 |
| EVFSTQADLSAITGTK | (SEQ ID NO:11) | 1666.86 | 1666.84 | 0.02 | 85 |
| AVLDVAETGTEAAAATGVK | (SEQ ID NO:12) | 1772.93 | 1772.92 | 0.01 | 42 |

| | | |
|---|---|---|
| | Protein | Sequence |
| Protein Hit: | Score: | coverage |

| | | |
|---|---|---|
| gi\|6678093\|ref\| serine proteinase inhibitor, clade A, member 3N | 428 | 23% |

| | | | | |
|---|---|---|---|---|
| | | Mr | Mr | |
| Peptide | (Observed) | (Expected) | Delta | Ion Score |

| | | | | | |
|---|---|---|---|---|---|
| VAQGIVSYGR | (SEQ ID NO:13) | 1048.56 | 1048.57 | −0.00 | 39 |
| HSHTLQEVK | (SEQ ID NO:14) | 1077.56 | 1077.56 | 0.01 | 21 |
| EQEPTQQFIPVK | (SEQ ID NO:15) | 1442.77 | 1442.74 | 0.03 | 76 |

| | | |
|---|---|---|
| | Protein | Sequence |
| Protein Hit: | Score: | coverage |

| | | |
|---|---|---|
| gi\|4758494\|ref\| granzyme B precursor (Homo sapiens) | 137 | 13% |

In all identified serpins, the part of the protein that interacts with the cognate protease is the reactive-center loop (RCL) (Whisstock et al., Trends Biochem. Sci. 23:63-67 (1998)). Table 2 shows the amino acid sequence of the RCL (P4-P4′ amino acids) of serpina3n and the other two serpins, the mouse SPI-6 and the human PI-9, which bind and inactivate granzyme B in mouse and humans, respectively (SEQ ID NO:16-18). Serpina3n sequence was directly compared to PI-9 to identify conserved residues and amino acid substitutions compatible with the binding to granzyme B (according to Sun et al., J. Biol. Chem. 276:15177-15184 (2001)).

TABLE 2

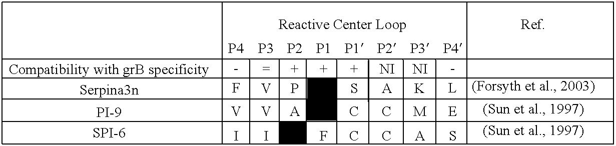

Gray cells indicate the hypothetical cleavage site for granzyme B (in between P1-P1′ residues).
The symbol “−” indicates an amino acid substitution in serpina3n (with respect to P1-9) that has a negative impact on the binding to granzyme B as assessed by scanning mutagenesis of P1-9 (Sun et al., J. Biol. Chem. 272: 15434-15441 (1997));
“=” is a conserved residue;
“+” indicates a conservative amino acid substitution in serpina3n that has been shown to be compatible with (P1) or to increase (P2 and P1′) granzyme B binding and cleavage;
“NI” indicates residues not critical for granzyme B binding.

Although granzyme B preferentially cleaves substrates at Asp or Glu residues (Thomberry et al., J. Biol. Chem. 272:17907-17911 (1997); Sun et al., J. Biol. Chem. 276:15177-15184 (2001)), it also cleaves after Met residues (Poe et al., J. Biol. Chem. 266:98-103 (1991); Odake et al., Biochemistry 30:2217-2227 (1991)). Therefore, the Met in the RCL of serpina3n (Table 2) may represent the P1 residue necessary for serpin cleavage by granzyme B. It is noteworthy that other residues in the reactive-center loop of serpina3n are conserved (or at least compatible) with the previously defined preferences of granzyme B for the interaction with PI-9 (Sun et al., J. Biol. Chem. 276:15177-15184 (2001)) (Table 2).

Serpina3n Forms a Covalent Complex with Granzyme B in Vitro

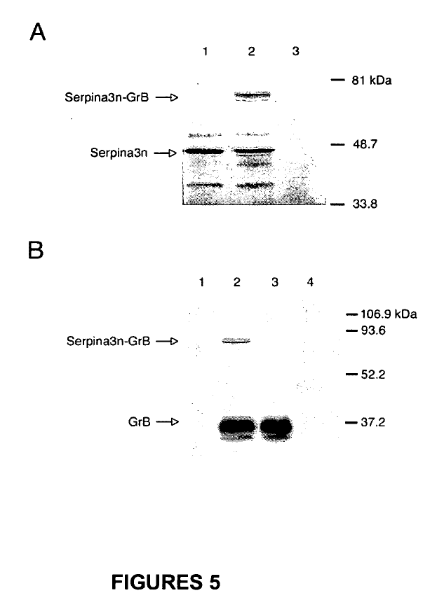

We then cloned a serpina3n cDNA from mouse liver total RNA by RT-PCR. As no anti-serpina3n antibody was available, an HA-tag at the serpin C-terminus was added to facilitate detection. The recombinant protein was transcribed/translated in vitro and tested for its ability to bind to purified granzyme B. As shown in FIGS. 5A and 5B, when granzyme B was added to the in vitro synthesized serpin, a high molecular weight complex of serpina3n with granzyme B formed, similar to the complex observed when granzyme B is incubated with SCCM. The slightly lower molecular weight of the complex formed by the recombinant serpin (with respect to the complex formed by the serpin secreted by Sertoli cells) is likely due to a lack of serpin glycosylation. These data confirmed that serpina3n is the protein secreted by Sertoli cells that binds to granzyme B.

Serpina3n Expressed in Jurkat Cells Is Secreted into the Medium and Inhibits Granzyme B Activity.

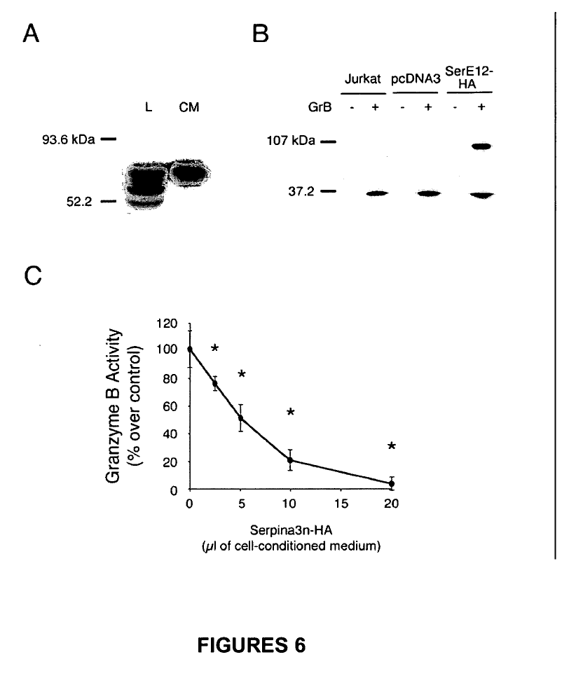

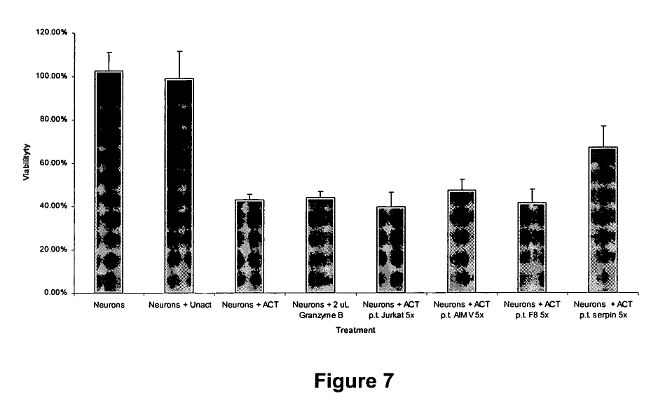

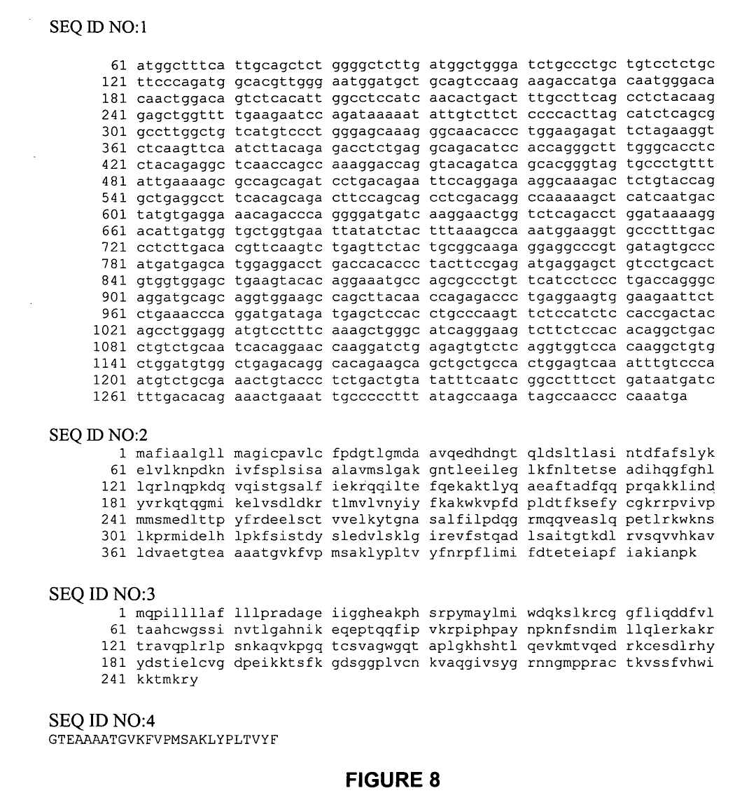

We next expressed serpina3n in Jurkat cells and selected stable clones with high transgene expression. FIG. 6A shows serpina3n expression in one of these clones, SerE12-HA, as well as its secretion into the culture medium. When the culture medium from the SerE12-HA clone was incubated with purified human granzyme B, a high molecular weight complex of serpina3n and granzyme B was formed (FIG. 6B). As expected, SerE12-HA conditioned medium also inhibited granzyme B enzymatic activity in a dose-dependent manner (FIG. 6C).

Serpina3n Protects Neurons against T-cell Mediated or Granzyme B Mediated Cell Death

We also have determined that T lymphocytes can mediate axonal and neuronal pathology in vitro and that serpina3n protects neurons from CTL-mediated cell death. Human fetal neurons in culture were treated with T lymphocytes isolated either from the peripheral blood of adult donors (allogeneic system) or from spleen of the same fetal specimen (syngeneic system). T lymphocytes, when activated by anti-CD3 treatment (but not when unactivated), killed neurons extensively. By 24 hours of co-culture, over 90% of neurons had degenerated. Moreover, T-cells aggregated around axons, leading to the rapid disappearance of microtubule associated protein-2 (MAP-2, a neuronal marker) and subsequent neuronal death. T-cell mediated killing of neurons occurred in either the allogeneic or syngeneic system, required activated T-cells, but did not require the presence of any exogenous antigen. Activated T lymphocytes can thus markedly affect the integrity of axons and neurons when they infiltrate the CNS in significant numbers.

As it has been previously shown that granzyme B can play a major role in T-cell-mediated neurodegeneration, the potential neuroprotective effect of serpina3n was examined. Activated T-cells were incubated for two hours with supernatant from Jurkat cells secreting serpina3n or incubated with a control (concentrated AIMV, concentrated Jurkat cell supernatant, or concentrated F8 supernatant). The T-cells were then cultured with human neurons. Twenty four hours later, a quantitative analysis of the neuronal viability was performed. Between 60% and 90% of neurons are lost in the co-culture with recombinant granzyme B, activated T-cells alone, or pre-treated with control supernatant. By contrast, only 30% of neurons were lost in the co-culture with activated T-cells pre-treated with serpina3n. Thus, serpina3n can be a neuroprotective agent and therefore may be useful in the treatment of inflammatory neuronal disorders (e.g., those described herein).

Materials and Methods

The following methods were used to perform the above-described experiments.

Animals, Cell Lines, and Reagents. Male BALB/c mice (University of Alberta, Edmonton, Alberta, Canada) were used as Sertoli cell donors.

L-cells (C3H mouse fibroblast cell line) were grown in Dulbecco's modified Eagle's medium (DMEM; Life Technologies, Burlington, Ontario) supplemented with 10% FBS, 2 mM L-glutamine, 100 U/ml penicillin and 50 μg/ml streptomycin (P/S). Mouse lymphocytic leukemia L1210 cells were maintained in RPMI 1640 medium supplemented with 20 mM HEPES, 50 U/ml penicillin, 50 μg/ml streptomycin, 1 mM sodium pyruvate (Life Technologies), 0.1 mM 2-mercaptoethanol (Sigma, St. Louis, Mo.) and 10% FBS. C57 cells (B6 mouse CTL cell line) were generated from splenocytes isolated from spleen of B6 mice that were stimulated with BALB/c or C3H mouse spleen cells. C57 cells were grown in RPMI 1640 (Life Technologies) supplemented with 10% FBS, 10⁻⁴ M 2-mercaptoethanol, 100 μg/ml P/S, 20 mM Hepes, and 80 units/ml of human recombinant IL2 (RHFM). Cells were maintained at a concentration of 5×10⁵ cells/ml and were stimulated once a week with irradiated BALB/c or C3H splenocytes (2500 rads) at a ratio of 1 (C57) to 14 (splenocytes).

Human granzyme B was purified from the cytolytic granules of YT INDY cells as described in Caputo et al., Proteins 35:415-424 (1999). Human replication deficient adenovirus (Adv) was prepared as previously described (Bett et al., Proc. Natl. Acad. Sci. USA. 91:8802-8806 (1994)). Mouse degranulate granzyme B material was prepared from CTL cells stimulated with immobilized anti-mouse CD3ε antibody (clone 145-2C11, BD Biosciences Pharmingen, San Diego, Calif.) as previously described (Sipione et al., J. Immunol. 174:3212-9 (2005)).

Isolation of Mouse Sertoli Cells and Preparation of Sertoli Cell-Conditioned Medium. Testicles were isolated from 9-12 day old male BALB/c mouse donors, and placed in HBSS containing 0.5% BSA (Sigma) on ice. Testicles were chopped and digested with collagenase (1 mg/ml; Sigma Type V) in a shaking water bath for six minutes at 37° C. The tissue was washed three times with HBSS and further digested with DNase (0.4 mg/ml, Boehringer Mannheim, Laval, Canada) and trypsin (1 mg/ml, Boehringer) in calcium-free medium containing 1 mmol/l EGTA and 0.5% BSA (Sigma) in a siliconized 250 ml flask in a shaking water bath for six minutes at 37° C. Following the second digest, the cells were washed with HBSS, filtered through a 500 μm nylon mesh, and then washed three more times before plating. Cell viability was determined by Trypan Blue exclusion. The number of GATA-4-positive Sertoli cells and smooth muscle alpha actin-positive peritubular myoid cells in culture was determined by immunohistochemistry as previously described (Dufour Gene Ther. 11:694-700 (2004)), using mouse monoclonal anti-GATA-4 (1:50; Santa Cruz Biotechnology, Santa Cruz, Calif.) and mouse monoclonal anti-smooth muscle alpha actin (1:50; DakoCytomation, Carpinteria, Calif.). In each preparation a minimum of 500 cells were counted.

For the preparation of conditioned medium, Sertoli cells were plated at the concentration of 5×10⁷ cells in 30 ml of serum-free HAM's F10 culture media supplemented with 0.5% BSA (no BSA was added when Sertoli cell-conditioned media was prepared for Western blot analysis), 100 U/ml penicillin and 100 U/ml streptomycin. The cells were cultured in tissue culture-treated plates for 3 days at 37° C. and 5% CO₂. The supernatant was then collected and spun in a centrifuge two times for 5 minutes each at 2000 RPM to remove cellular debris. The resulting Sertoli cell-conditioned media (SCCM) was then concentrated with an Amicon YM-10 Centricon device (molecular weight cut-off of 10 kDa; Fisher Scientific, Ottawa, Ontario) for 90 minutes at 7000 RPM (4° C.) to a volume of 3 ml (10× concentration). Serum-free HAM's F10 with or without 0.5% BSA, was concentrated in a similar manner to be used as a control medium. Protein concentration was determined with Bradford protein assay (BioRad Laboratories, Hercules, Calif.). SCCM was stored at 4° C. until used.

CTL Killing Assay. ³H-thymidine labeled L1210 cells were pre-incubated with HAM's F-10 control media or SCCM for one hour at 37° C. C57 effector cells were then mixed with L1210 cells at a ratio of 10:1 (effector to target cell ratio) and incubated for 3 hours at 37° C. Following the 3 hour incubation, samples of target and effector cells were prepared for determination of ³H-thymidine release. Sample lysis buffer (1% Triton X, 200 μl) was added to each eppendorf tube containing the samples and tubes were mixed for one minute using a vortex machine. Tubes were subsequently spun at 1400 RPM for 10 minutes at 4° C. Supernatants were transferred to liquid scintillation vials and aqueous counting scintillant was added. Samples were then placed in a beta counter for the determination of amount of ³H-thymidine release. The percent specific ³ H-thymidine release per sample was calculated as follows: [(sample count [target and effector]−spontaneous count [target alone])/(totals count−spontaneous count)]×100.

Granzyme B-Mediated Apoptosis and TUNEL Assay. Fibroblast L-cells were seeded into a 96-well plate at a concentration of 2×10⁵ cells/well and pre-incubated with 25 μl of concentrated SCCM or HAM's F10 (control) for 30 minutes at 37° C. Increasing concentrations of human granzyme B and 100 pfu/well of adenovirus, adenovirus alone or granzyme B alone were added to the cells. Cells were incubated for three hours at 37° C., washed with phosphate buffered saline (PBS) supplemented with 2% FBS, and fixed with 2% paraformaldehyde and 1% FBS overnight at 4° C. A TdT-mediated dUTP nick end labeling (TUNEL) assay was used to measure the amount of DNA fragmentation, a hallmark feature of apoptosis that occurs in target cells upon incubation with granzyme B. Following the overnight fixation procedure, L-cells were washed 3 times with PBS/2% FBS and permeabilized with 0.1% saponin in PBS for one hour at room temperature. Cells were then washed 3 times with PBS/2% FBS and incubated with TUNEL mix (20 μl, Roche Diagnostic, Laval, Quebec) and incubation for 1.5 hours at 37° C. Following two washes in PBS/2% FBS the cells were resuspended in PBS/2% FBS and analyzed with a fluorescence activated cell sorter (FACS, FACScan, BD Biosciences) to derive the percentage of TUNEL positive cells.

Mannose-6 Phosphate Receptor Expression and Granzyme B Uptake. L-cells were added to 96-well plates at a concentration of 2×10⁵ cells/well and pre-incubated with SCCM or HAM's F-10 control media for one hour at 37° C. For CI-MPR and CD-MPR staining, L-cells were incubated for one hour at 4° C. with PBS (0.1% BSA, control), rabbit anti-bovine CI-MPR (1/500, William Brown, Cornell University), or rabbit anti-human CD-MPR (1/100, William Sly, Saint Louis University), both of which cross-react with the mouse proteins (Motyka et al., Cell 103:491-500 (2000)). After washing, cells were incubated for 20 minutes at 4° C. with goat anti-rabbit conjugated to Fluorescein Isothiocyanate (FITC, 1/100, Jackson, Mississauga, Ontario). Cells were then washed with PBS supplemented with 2% FBS and fixed in PBS with 2% paraformaldehyde and 1% FBS (180 μl) overnight at 4° C. The after cells were then washed several times with PBS/2% FBS prior to being acquired and analyzed with a fluorescence activated cell sorter (FACS scan, BD Biosciences).

For the detection of granzyme B binding and uptake, L-cells were added to 96-well plates at a concentration of 2×10⁵ cells/well and pre-incubated with SCCM or HAM's F-10 control media for one hour at 37° C. For granzyme B binding to L-cells, cells were incubated for one hour at 4° C. with PBS (0.1% BSA) and granzyme B conjugated to Alexa 488 (Molecular Probes). Cells were then washed with PBS and fixed as described above before performing FACS analysis. For granzyme B uptake into L-cells, cells were incubated for one hour at 37° C. with DMEM (0.1% BSA) and granzyme B conjugated to Alexa 488. Cells were then washed with DMEM with 0.1% BSA, fixed and analysed by FACS.

Granzyme B Enzymatic Activity Assay. Isoleucine/glutamate/proline/aspartate conjugated to paranitroanalide (IEPD-pNA) contains the cleavage site for granzyme B. When IEPD-pNA is cleaved by granzyme B it produces IEPD and pNA, a colored product, whose absorbance can be measured at 405 nm and assumed to be proportional to the amount of granzyme B enzymatic activity in the assay.

Human purified granzyme B and mouse CTL degranulate granzyme B were incubated with PBS/2% FBS, HAM's F-10 media, or SCCM for 30 min at 37° C. in 96-well plates. Granzyme B enzymatic activity was then measured as previously described (Ewen et al., J. Immunol. Methods 276:89-101 (2003)). Briefly, a reaction mix containing 50 mM HEPES, pH 7.5, 10% (w/v) sucrose, 0.05% (w/v) CHAPS, 5 mM DTT and 200 μM Acetyl-Ile-Glu-Pro-Asp-paranitroanilide (Ac-IEPD-pNA) (Kamiya Biomedical, Seattle, Wash.) was added to each sample. The plate was then incubated for 5 hours at 37° C. Hydrolysis of Ac-IEPD-pNA was measured at 405 nm at time zero and every hour thereafter, using a Multiskan Ascent spectrophotometer (Thermo Lab-System, Helsinki, Finland).

Western Blottingfor Granzyme B and SPI-6. Granzyme B (36 ng) was incubated with 40 μl of concentrated SCCM (BSA-free), with the same amount of concentrated HAM's F-10 medium or with PBS for 2 hours at 37° C. SDS sample buffer was added to the samples which were then denatured by heating at 100° C. for 5 minutes. Proteins were separated on a 10% SDS-polyacrylamide gel at 30 mA/gel for 1.5 hours and transferred to a PVDF membrane (Millipore, Bedford, Mass.).

Immunodetection of granzyme B was performed with a mouse monoclonal anti-human granzyme B antibody (clone 2C5, 1:500 dilution, Santa Cruz, Santa Cruz, Calif.). The secondary antibody used was an anti-mouse horse radish peroxidase-conjugated antibody (1:3000, Bio Rad, Mississauga, Ontario). SPI-6 immunodetection was performed with two different antibodies, a rabbit anti-mouse SPI-6 antibody (1:5000 dilution, kindly provided by Dr. J. P. Medema, Leiden University Medical Center, Leiden, The Netherlands) and a mouse anti-human PI-9 antibody (P19-17, 8.5 μg/ml, Alexis Biochemicals, San Diego, Calif.) known to cross-react with SPI-6 (Bladergroen et al., J. Immunol. 3218-3225 (2001); Medema et al., J. Exp. Med. 194:657-667 (2001)). An anti-rabbit horse radish peroxidase-conjugated antibody (1:20000, Bio Rad) or an anti-mouse horse radish peroxidase-conjugated antibody (1:3000, Bio Rad) were used as secondary antibodies, respectively. Detection of immunoreactive bands was performed by ECL Plus (Amersham Biosciences, Piscataway, N.J.). Where indicated, PVDF membranes were stripped with 62.5 mM Tris-HCl (pH 6.7) containing 2% SDS and 100 mM 2-mercaptoethanol for 30 minutes at 60° C. in a shaking water bath, before re-probing with a different antibody.

Granzyme B Immunoprecipitation and Characterization of the Serpin-Granzyme B Complex. Human granzyme B (1 μg) was incubated for 2 hours at 37° C. with 1 ml of SCCM previously concentrated as indicated above. Pre-clearing of the sample was performed by adding 1 ml of PBS containing 1% NP-40 and 0.5% Na deoxycholate (binding buffer) and 100 μl protein G-Sepharose (2 mg protein G/ml drained medium; Amersham Biosciences Corp., Piscataway, N.J., USA) for one hour at 4° C. Immunoprecipitation of granzyme B was carried out overnight at 4° C. with a monoclonal anti-human granzyme B antibody (clone 2C5, Santa Cruz, Calif.) followed by incubation with protein G-Sepharose for three hours at 4° C. The immunoprecipitate was washed three times with binding buffer and four times with PBS, resuspended in SDS sample buffer, and denatured at 100° C. for 10 min. The immunoprecipitated proteins were resolved by SDS-PAGE and protein bands in the gel were revealed by Coomassie blue R staining. Small aliquots of the sample, collected before and after immunoprecipitation, were run on the same gel and transferred onto PVDF membrane. Western blot for granzyme B was performed as indicated above and compared to the pattern of bands revealed by Coomassie blue-staining of the gel. The band in the gel that matched the high molecular weight-immunoreactive band in the Western blot was excised and analyzed by MALDI-TOF mass spectrometry at the Institute for Biomolecular Design (IBD, University of Alberta, Canada). Briefly, an automated in-gel tryptic digestion was performed on a Mass Prep Station (Micromass, UK). The gel pieces were de-stained, reduced (DTT), alkylated (iodoacetamide), digested with trypsin (Sequencing Grage, Promega) and the resulting peptides extracted from the gel and analyzed via LC/MS/MS. LC/MS/MS was performed on a CapLC HPLC (Waters, USA) coupled with a Q-ToF-2 mass spectrometer (Waters, USA). Tryptic peptides were separated using a linear water/acetonitrile gradient (0.2% formic acid) on a Picofrit reversed-phase capillary column, (5 micron BioBasic C18, 300 Angstrom pore size, 75 μm ID×10 cm, 15 μm tip) (New Objectives, Mass., USA), with an in-line PepMap column (C18, 300 μm ID×5 mm), (LC Packings, Calif., USA) used as a loading/desalting column.

Protein identification from the generated MS/MS data was done searching the NCBI non-redundant database using the Mascot search engine (Mascot Daemon, Matrix Science, UK) at www.matrixscience.com, with stringency of 0.6 Da. Search parameters included carbamidomethylation of cysteine, possible oxidation of methionine, and one missed cleavage per peptide.

Cloning and Expression of Serpina3n. Hemaegglutinin (HA)-tagged serpina3n (serpina3n-HA) was cloned by RT-PCR from mouse liver total RNA, using Superscript II and Platinum Taq DNA polymerase (Invitrogen, Carlsbad, Calif., USA), according to the manufacturer's instructions. The serpina3n cDNA was amplified with the following specific primers: 5′-CGCGGATCCATGGCTTTCATTGCAGCTCTGG-3′ (forward) (SEQ ID NO:19) and 5′-CGCCTCGAGTCAGGCGTAGTCGGGGACGTCGTAGGGGTAGAATTTGGGGTTC GCTATCTTGGC-3′ (reverse) (SEQ ID NO:20). The forward primer included a BamHI restriction site and the reverse primer included a XhoI restriction site for subsequent cloning. The reverse primer also included a short sequence coding for HA-tag at the carboxy-terminal of the serpin. The cDNA was digested with BamHI and XhoI restriction enzymes and cloned into pcDNA3 vector (Invitrogen).

Jurkat cells were electroporated with serpina3n-HA-pcDNA3 and single neomycin-resistant cells were sorted by FACS for clonal expansion. Expression of 0serpina3n-HA in the transfected clones was verified by immunoblotting with anti-HA antibody (Clone H A. 11, 1:1000, Covance Research Products, Cumberland, Va., USA).

In Vitro Binding of Serpina3n-HA to Human Granzyme B. Radiolabeled (35S-methionine) serpina3n-HA protein was produced in vitro using TNT® Coupled Reticulocyte Lysate Systems (Promega, Madison, Wis., USA) according to manufacturer's instructions. One (1) μg DNA was used for each reaction. Two (2) μl of the reaction volume were incubated with purified human granzyme B in PBS for 30 minutes at room temperature. Samples were then resolved by SDS-PAGE and visualized by autoradiography and by immunoblotting for granzyme B as indicated above.

Preparation of Serpina3n-Containing Medium. Jurkat cell clones expressing serpina3n-HA and control cells transfected with pcDNA3 vector were incubated overnight in Opti-MEM I (Invitrogen) at 5×10⁶ cells/ml. Cell-conditioned medium was concentrated to ⅕^{th} of its original volume using Amicon YM-10 Centricon filters, as described above and was used immediately for experiments.

Preparation of Human Fetal Neurons. Human fetal neurons were used as the targets of neurotoxicity studies in culture, as it has not been possible to isolate and maintain the survival of neurons from adult human brain specimens. Human fetal neurons were cultured from specimens obtained by therapeutic abortion. The gestational age of the specimens ranges from 15 to 20 weeks. To obtain neurons, brain tissue was diced into fragments. The suspension was then filtered and centrifuged. The pellet was suspended in PBS, and after a final wash in feeding medium, the cells were plated into T-75 flasks. To obtain a neuron-enriched culture, cells in the flasks were treated with cytosine arabinoside to kill the dividing astrocytes. In this way, neuronal cultures in excess of 90% purity and less than 5% astrocytes, are generated, which were then plated in 16-well Lab-tek slides. T lymphocytes were isolated from the peripheral blood of adult healthy donors by Ficoll-Hypaque centrifugation and suspended in serum-free AIM-V medium. To activate T-cells, 1 μg/ml of an anti-CD3 antibody (OKT3) were added once for a period of 3 days. The floating cells were then removed from any adherent monocytes, and a fixed density was then used for testing cytotoxicity. Unactivated T-cells are prepared in the absence of OKT3. These cells are subject to centrifugation, and the floating cells collected 3 days later. Flow cytometry analyses of the floating cells collected after 3 days of initiation of OKT3 treatment indicated that CD3⁺T-cells constitute over 90% of the total cell population; these are approximately 60% CD4⁺ and 40% CD8⁺ in cell ratio. B lymphocytes (CD19⁺) and NK cells (CD56⁺) consist of the rest of the floating cell population; no monocytes (CD14⁺) are detected. NK cells are found to constitute <3% of the population. There is no significant difference in the proportion of the various cell subsets between the unactivated and activated lymphocyte populations.

Statistics. Statistical significance of differences between two independent groups was calculated with a paired Student's t-test. A value of p<0.05 was considered significant.

Cells Containing a Polynucleotide Encoding a Granzyme B Inhibitory Serpin

The invention provides a cell containing a heterologous polynucleotide encoding a granzyme B inhibitory serpin (e.g., serpina3n). Those skilled in the field of molecular biology will understand that any of a wide variety of cellular systems may be used to provide cells of the invention. Cells may include, for example, eukaryotic cells such as Saccharomyces cerevisiae, insect cells (e.g., Sf21 cells), or mammalian cells (e.g., Brockmann bodies, Sertoli, islet, NIH 3T3, HeLa, or COS cells). Such cells are available from a wide range of sources (e.g., the American Type Culture Collection, Rockland, Md.; also, see, e.g., Ausubel et al., Current Protocols in Molecular Biology, Wiley Interscience, New York, 2000; PCR Technology: Principles and Applications for DNA Amplification, ed., H. A. Ehrlich, Stockton Press, N.Y.; and Yap and McGee, Nucl. Acids Res. 19:4294 (1991)). The method of transformation or transfection and, if desired, the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

Compositions of cells, where one or more cells of the composition include a heterologous polynucleotide coding for a granzyme B inhibitory serpin (e.g., serpina3n) are also provided by the present invention. In one example, a composition of the invention includes a Sertoli cell and an islet cell. In this example, either one or both of the cells may contain a polynucleotide encoding a granzyme B inhibitory serpin (e.g., serpina3n), and may express a granzyme B inhibitory serpin such as serpina3n. In certain embodiments of the invention, a cell both expresses and secretes serpina3n. Cells and cell compositions of the invention may contain an additional heterologous polynucleotide. In one embodiment, a non-human cell (e.g., a pig cell) may be altered to contain two heterologous polynucleotides, one polynucleotide encoding a granzyme B inhibitory serpin, the second polynucleotide encoding human insulin. Such a cell may be used in the methods of the invention, for example, by introducing the cell into a patient (e.g., a human) with a disease such as diabetes (e.g., diabetes type I).

Generation of Novel Granzyme B Inhibitory Serpins

Chimeric polypeptides with granzyme B inhibitory activity may be generated from the compositions and methods of the present invention using molecular biological techniques standard in the art (e.g., those described in Ausubel et al., supra).

As noted above, serpina3n is a member of a multigene family of serpins with high degree of homology with the human α1-antichymotrypsin (SERPINA3). The interaction of these serpins is mediated primarily through the reactive center loop (e.g., the specificity of serpina3n for granzyme B); it therefore is possible to generate chimeric serpin polypeptides (e.g., chimeric human α1-antichymotrypsin polypeptides) that specifically bind granzyme B. Granzyme B inhibitory activity can be assayed for using methods known in the art or those described herein. It can be desirable to generate such chimeric polypeptides, for example, to decrease antigenicity of a polypeptide (e.g., a antigenicity of a polypeptide when administered to a human patient) using methods of the invention. In one example, a human α1-antichymotrypsin polypeptide containing the reactive center loop sequence of serpina3n can be generated. In certain embodiments, the novel granzyme B inhibitory serpin contains a sequence that targets the serpin for secretion from a cell (e.g., a cell producing the granzyme B inhibitory serpin). Such sequences are known in the art and include the amino-terminal secretory sequence present in serpina3n.

Fragments of granzyme B inhibitory serpins may also be useful in the methods and compositions of the invention. Particularly useful fragments may include those with the serpina3n RCL. Granzyme B inhibitory activity of serpin fragments may be assayed using methods known in the art or those described herein.

Therapeutic Methods Employing Granzyme B Inhibitory Serpin Polynucleotides and Polypeptides

The invention includes methods of treating a patient in need of immunosuppressive therapy by using an immunosuppressive agent such as a granzyme B inhibitory serpin (e.g., serpina3n).

A granzyme B inhibitory serpin (e.g., serpina3n) or a granzyme B binding fragment or analog thereof that exhibits immunosuppressive activity are considered particularly useful in the invention. Such polypeptides may be used, for example, as therapeutics to decrease the CTL mediated killing of islet cells in a individual with diabetes. Other immunological disorders that may be treated using an immunosuppressive agent, or an agent that reduces the immune function are described herein and include acute inflammation, rheumatoid arthritis, allergic reactions, asthmatic reactions, inflammatory bowel diseases (e.g., Crohn's Disease and ulcerative colitis), transplant rejection, inflammatory vascular diseases, inflammatory neuronal diseases, and restenosis.

Treatment or prevention of diseases resulting from an immune disorder (e.g., any autoimmune disorder described herein such as diabetes or rheumatoid arthritis), an inflammatory vascular disease, an inflammatory neuronal disease, or resulting from a cell (e.g., an organ) transplantation is accomplished, for example, by decreasing the activity of granzyme B by delivering, for example, a granzyme B inhibitory serpin (e.g., serpina3n) to an appropriate cell (e.g., an islet cell).

Direct administration of a recombinant granzyme B inhibitory serpin (e.g., serpina3n) polynucleotide or polypeptide, either to the site of a potential or actual disease-affected tissue or transplanted tissue (for example, by injection), or systemically for treatment of, for example, an autoimmune disease (e.g., diabetes or rheumatoid arthritis), an inflammatory vascular disease, or an inflammatory neuronal disease, can be performed accordingly to any conventional recombinant protein administration technique known in the art or described herein. The actual dosage depends on a number of factors known to those of ordinary skill in the art, including the size and health of the individual patient, but generally, between 0.1 mg and 100 mg inclusive are administered per day to an adult in any pharmaceutically-acceptable formulation. Such formulations are described herein.

Gene Therapy

Gene therapy is another therapeutic approach for expressing a granzyme B inhibitory serpin (e.g., serpina3n) in a patient. Heterologous nucleic acid molecules, encoding, for example serpina3n, a biologically active fragment of serpina3n, or a serpina3n fusion protein, can be delivered to the target cell of interest. The nucleic acid molecules must be delivered to those cells (e.g., islet cells) in a form in which they can be taken up by the cells and so that sufficient levels of protein can be produced to suppress an immune response.

Transducing viral (e.g., retroviral, adenoviral, and adeno-associated viral) vectors can be used for somatic cell gene therapy, especially because of their high efficiency of infection and stable integration and expression (see, e.g., Cayouette et al., Hum. Gene Ther. 8:423-430 (1997); Kido et al., Curr. Eye Res. 15:833-844 (1996); Bloomer et al., J. Virology 71:6641-6649 (1997); Naldini et al., Science 272:263-267 (1996); and Miyoshi et al., Proc. Natl. Acad. Sci. USA 94:10319-10323 (1997)). For example, a full length gene, or a portion thereof, can be cloned into a retroviral vector and expression can be driven from its endogenous promoter, from the retroviral long terminal repeat, or from a promoter specifically expressed in a target cell type of interest (e.g., a Sertoli cell or an islet cell). Other viral vectors that can be used include, for example, a vaccinia virus, a bovine papilloma virus, or a herpes virus, such as Epstein-Barr Virus (also see, for example, the vectors of Miller, Human Gene Therapy 15-14 (1990); Friedman, Science 244:1275-1281 (1989); Eglitis et al., BioTechniques 6:608-614 (1988); Tolstoshev et al., Curr. Opin. Biotechnol. 1:55-61 (1990); Sharp, Lancet 337:1277-1278 (1991); Cometta et al., Nuc. Acid Res. Mol. Biol. 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med. 323:370 (1990); U.S. Pat. No. 5,399,346).

Non-viral approaches can also be employed for the introduction of therapeutic nucleic acids to target cells of a patient. For example, a nucleic acid molecule (e.g., encoding a granzyme B inhibitor serpin such as serpina3n or a fragment thereof) can be introduced into a cell by administering the nucleic acid in the presence of lipofection (Felgner et al., Proc. Natl. Acad. Sci. USA 84:7413 (1987); Ono et al., Neurosci. Lett. 17:259 (1990); Brigham et al., Am. J. Med. Sci. 298:278 (1989); Staubinger et al., Meth. Enzymol. 101:512 (1983)), asialoorosomucoid-polylysine conjugation (Wu et al., J. Biol. Chem. 263:14621 (1988); Wu et al., J. Biol. Chem. 264:16985 (1989)), or by micro-injection injection under surgical conditions (Wolff et al., Science 247:1465 (1990)). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in gene therapy methods can be directed from any suitable promoter (e.g., the early immediate promoter of the human cytomegalovirus, CMV) and regulated by any appropriate mammalian regulatory element. In cases where inducible expression is desired, inducible promoters, such as tetracycline-responsive minimum essential CMV promoter coupled to a constitutively active promoter (e.g., a glycerol-3-phosphate dehydrogenase (GPDH) promoter) may be used. Such a system would be useful, for example, when high level expression of a granzyme B inhibitory serpin is desired initially in a cell (e.g., an islet cell), but lower expression levels are desired some time later. It may be desired to limit, for example, granzyme B inhibitory serpin expression to tissue or a spatial region in which an immunosuppression is desired. In one example, an enhancer known to preferentially direct gene expression in an islet cell can be used to direct the expression of a nucleic acid that encodes serpina3n. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

A desired mode of gene therapy is to provide the polynucleotide in such a way that it will replicate inside the cell, enhancing and prolonging the desired effect. Thus, the polynucleotide is operably linked to a suitable promoter, such as the natural promoter of the corresponding gene, a heterologous promoter that is intrinsically active in target cell, or a heterologous promoter that can be induced by a suitable agent.

Transgenic Animals

The present invention also includes the use of transgenic animals (e.g., mice, rats, pigs, and fish) expressing a gene encoding an exogenous granzyme B inhibitory serpin. Such animals may be used as a source of tissue or cells for transplantation into a patient. Particularly useful are islet cells from a transgenic pig or Brockmann bodies from a transgenic fish expressing a granzyme B inhibitory serpin such as serpina3n. In one example, cells from a non-human animal (e.g., a pig) expressing both a granzyme B inhibitory serpin (e.g., serpina3n) and human insulin may be used for transplantation into a patient in treatment of diabetes.

Construction of transgenes can be accomplished using any suitable genetic engineering technique, such as those described in Ausubel et al. (supra). Many techniques of transgene construction and of expression constructs for transfection or transformation in general are known and may be used for the disclosed constructs.

One skilled in the art will appreciate that a promoter is chosen that directs expression of a polynucleotide in a desired tissue. For example, as noted above, any promoter that regulates expression of a nucleic acid sequence described herein can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). One skilled in the art would also know how to make a transgenic mouse or rat. A transgenic pig may be generated using the method described in Velander et al. (Proc. Natl. Acad. Sci. USA 89, 12003-12007 (1992)).

Pharmaceutical Compositions for Decreasing Granzyme B Activity

The present invention includes the administration of a granzyme B inhibitory serpin (e.g., serpina3n) or granzyme B inhibitory fragment thereof for the treatment of a patient in need of immunosuppressive therapy. The administration of any granzyme B inhibitory serpin (e.g., serpina3n or a granzyme B binding fragment thereof), regardless of its method of manufacture, may provide granzyme B inhibitory biological activity in a patient with undesired or excessive CTL activity that occurs, for example, in an autoimmune disorder (e.g., diabetes or rheumatoid arthritis), an inflammatory vascular disease, or an inflammatory neuronal disease.

For example, a granzyme B inhibitory serpin (e.g., serpina3n) can be administered to a patient, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (19th edition), ed. A. Gennaro, 1995, Mack Publishing Company, Easton, Pa.

Pharmaceutical formulations of a therapeutically effective amount of a granzyme-B inhibitory serpin polypeptide, polynucleotide, or a fragment thereof, or pharmaceutically acceptable salt-thereof, can be administered orally, parenterally (e.g., intramuscular, intraperitoneal, intravenous, or subcutaneous injection), or by any other route in an admixture with a pharmaceutically acceptable carrier adapted for the route of administration.

Methods well known in the art for making formulations are found, for example, in Remington's Pharmaceutical Sciences, (19th edition), ed. A. Gennaro, 1995, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The amount of active ingredient in the compositions of the invention can be varied. One skilled in the art will appreciate that the exact individual dosages may be adjusted somewhat depending upon a variety of factors, including the protein being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

Granzyme B inhibitory serpin (e.g., serpina3n) polypeptides, polynucleotides, or any vehicle that includes such polypeptides or polynucleotides can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or subacute disorder, a treatment with an immediate release form will be preferred over a prolonged release composition. Alternatively, for preventative or long-term treatments, a sustained released composition will generally be preferred.

A pharmaceutical composition containing, for example, a serpina3n polypeptide, serpina3n polynucleotide, or a fragment thereof, can be prepared in any suitable manner. The protein or therapeutic compound can be isolated from naturally occurring sources, recombinantly produced, or produced synthetically, or produced by a combination of these methods. The synthesis of short peptides is well known in the art. See, e.g., Stewart et al., Solid Phase Peptide Synthesis (Pierce Chemical Co., 2^{nd} ed., 1984).

Cell Transplantation

The invention also provides methods for treating patients in need of immunosuppression by transplantation of a cell containing a polynucleotide encoding a granzyme B inhibitory serpin (e.g., a cell expressing serpina3n). Methods of the invention may include allogeneic (between genetically different members of the same species), autologous (transplantation of an organism's own cells or tissues), syngeneic (between genetically identical members of the same species (e.g., identical twins)), or xenogeneic (between members of different species) transplantation. Methods of the invention include, for example, administering to the patient islet cells, and combinations of cells that include islet and a second cell (e.g., Sertoli cells expressing a granzyme B inhibitory serpin). Transplantation of the cells of the invention into a patient in need of immunosuppression will result in a decreased immune response, which may effect treatment of an autoimmune disorder such as rheumatoid arthritis, or, in the case of diabetes, may serve to prevent co-transplanted insulin-producing cells such as islet cells from an undesired immune response. In other embodiments, the transplanted cells may effect treatment of an inflammatory vascular disease or an inflammatory neuronal disease. The cells are introduced into a patient in need of immunosuppression in an amount suitable to effect a reduction of at least one immune response. The cells can be administered to a patient by any appropriate route that results in delivery of the cells to a desired location in the patient where at least a portion of the cells remain viable. It is desirable that at least about 5%, desirably at least about 10%, more desirably at least about 20%, yet more desirably at least about 30%, still more desirably at least about 40%, and most desirably at least about 50% or more of the cells remain viable after administration into a patient. The period of viability of the cells after administration to a patient can be as short as a few hours, e.g., twenty-four hours, to a few days, to as long as a few weeks to months. Due to the chronic nature of many autoimmune disorders, it is desired that transplanted cells remain viable for months or years following transplantation. The transplanted cells can be administered in a physiologically compatible carrier, such as a buffered saline solution.

To perform these methods of administration, the cells of the invention can be inserted into a delivery device that facilitates introduction by injection or implantation of the cells into the patient. Such delivery devices include tubes, e.g., catheters, for injecting cells and fluids into the body of a recipient patient.

In a preferred embodiment, the tubes additionally have a needle or needles through which the cells of the invention can be introduced into the patient at a desired location (e.g., in the kidney capsule, liver, omental pouch). In an embodiment where multiple types of cells are transplanted, it may be desirable to maintain the different cell types in a different set of conditions (such as in different media) during the injection.

The cells used in methods of the invention can be inserted into such a delivery device in different forms. For example, the cells can be suspended in a solution or embedded in a support matrix (e.g., alginate microcapsule) when contained in such a delivery device. Preferably, the solution includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, or thimerosal. Solutions used in the invention can be prepared by incorporating the cells as described herein in a pharmaceutically acceptable carrier or diluent and, as required, other ingredients.

Support matrices in which the cells of the invention can be incorporated or embedded include matrices which are recipient-compatible and which degrade into products that are not harmful to the recipient. Natural and/or synthetic biodegradable matrices are examples of such matrices. Natural biodegradable matrices include, for example, collagen matrices and alginate beads. Synthetic biodegradable matrices include synthetic polymers such as polyanhydrides, polyorthoesters, and polylactic acid. These matrices provide support and protection for the cells in vivo.

Prior to introduction into a patient, the cells can be further modified to inhibit immunological rejection. For example, to inhibit rejection of transplanted cells and to achieve immunological non-responsiveness in a transplant recipient, a method of the invention can include alteration of immunogenic antigens on the surface of the cells prior to introduction into the patient. This step of altering one or more immunogenic antigens on the cells can be performed alone or in combination with administering to the patient an agent that inhibits CTL cell activity in the patient. Alternatively, inhibition of rejection of the transplanted cells can be accomplished by administering to the patient an agent that inhibits T-cell activity (e.g., serpina3n or other immunosuppressant described herein) in the patient in the absence of prior alteration of an immunogenic antigen on the surface of the transplanted cells. An agent that inhibits CTL cell activity is defined as an agent which results in removal (e.g., sequestration) or destruction of CTL cells within a patient or inhibits CTL cell functions within the patient. CTL cells may still be present in the patient but are in a non-functional state, such that they are unable to proliferate or elicit or perform effector functions (e.g., cytokine production, cytotoxicity, etc). The agent that inhibits T-cell activity may also inhibit the activity or maturation of immature T-cells (e.g., thymocytes). A preferred agent for use in inhibiting T-cell activity in a recipient patient is an immunosuppressive drug that inhibits or interferes with normal immune function. A exemplary immunosuppressive drug is cyclosporin A. Other immunosuppressive drugs that can be used include, for example, Tacrolimus (FK506, Prograff), Sirolimus (Rapamune), Daclizumab, Mycophenolate Mofetil (RS-61443, CellCept), or antibodies (e.g., monoclonal antibodies) specific for CTL cells. In one embodiment, the immunosuppressive drug is administered in conjunction with at least one other therapeutic agent. Additional therapeutic agents that can be administered include steroids (e.g., glucocorticoids such as prednisone, methyl prednisolone, and dexamethasone) and chemotherapeutic agents (e.g., azathioprine and cyclosphosphamide) and monoclonal antibodies. In another embodiment, an immunosuppressive drug is administered in conjunction with both a steroid and a chemotherapeutic agent. Suitable immunosuppressive drugs are commercially available.

Sources of Cells for Transplantation

Living islet donors. Current islet transplantation protocols rely on cadaveric pancreas donors as a source of islets for transplantation (Shapiro et al., Immunol. Rev. 196:219-236 (2003)). At present, the pool of cadaveric pancreata available for islet isolation and transplantation is limited and alternative sources are desirable to allow for the widespread use of islet transplantation for the treatment of diabetes. Some centers have had success using living donors for simultaneous pancreas and kidney transplantation (Gruessner et al., Transplant. Proc. 30:282 (1998); Benedetti et al., Transplantation 67:915-918 (1999); Zielinski et al., Transplantation 76:547-552 (2003)). This procedure involves the removal of a portion of the living donor pancreas for transplantation into the diabetic recipient. This technique may be extended to islet transplantation. Procurement of organs from living donors is advantageous as the quality of the organ isolated from a living donor should be greatly improved as compared to an organ isolated from a brain-dead donor (Gruessner et al., Transplantation 61:1265-1268 (1996)). Additionally, HLA matching between donor and recipient can occur if the donor is, for example, a living relative. By obtaining a closer immunologic match, the amount of immunosuppression required may be reduced and there may be improved the function and lifetime of the transplanted organ (Cicalese et al., Int. Surg. 84:305-312 (1999)). Finally, such an approach to organ procurement offers the advantage of reducing waiting time and possibly percentage of deaths of people on the transplant list.

Beta cell lines. Another potential source of tissue is pancreatic β-cell lines (Efrat et al., Ann. N. Y. Acad. Sci. 875:286-293 (1999)). The formation of a β-cell line requires the creation of an immortalized β-cell that has been oncogenically transformed. For example, βTC cell lines have been created using transgenic technology whereby transgenic mice harboring the SV40 T-antigen driven by the insulin gene enhancer-promoter region develop heritable β-cell tumors (Hanahan, D., Nature 315:115-122 (1985); Efrat et al., Proc. Natl. Acad. Sci. USA 85:9037-9041 (1988); Miyazaki et al., Endocrinology 127:126-132 (1990); Hamaguchi et al., Diabetes 40:842-849 (1991)). The mouse β-cell lines have been reported to produce insulin in amounts comparable to normal islets and release insulin in response to physiological stimuli (Efrat et al., Ann. N. Y. Acad. Sci. 875:286-293 (1999)). These cell lines also normalize glycemia in diabetic mice (Efrat et al., Proc. Natl. Acad. Sci. USA 92:3576-3580 (1995)).

Stem cells. One potential source of insulin secreting tissue for transplantation into patients with type 1 diabetes may come from stem cells (Street et al., Curr. Top. Dev. Biol. 58:111-136 (2003)). Stem cells are self-renewing elements that can generate the many cell types in the body. They are found in adult and fetal tissues, but the stem cells with the widest developmental potential are derived from an early stage of the mammalian embryo and are called embryonic stem (ES) cells. ES cells have been shown to differentiate in vitro into many different cell types including pancreatic islet-like structures ((Wiles et al., Development 111:259-267 (1991); Rohwedel et al., Dev. Biol. 164:87-101 (1994); Wobus et al., Differentiation 48:173-182 (1991); Dani et al., J. Cell Sci. 110(Pt 11):1279-1285 (1997); Okabe et al., Mech. Dev. 59:89-102 (1996); Abe et al. Exp. Cell Res. 229:27-34 (1996)). Lumelsky et al., operating under the assumption that a strategy used to generate neurons would lead to the development of islet-like structures, cultured mouse ES cells in vitro under conditions that enriched in cells expressing the neural stem cell marker nestin (Lumelsky et al., Science 292:1389-1394 (2001)). These nestin positive cells were further differentiated into structures morphologically resembling islets. Further studies have improved upon the original protocol and have been able to generate cells that can correct hyperglycemia in diabetic animals (Hori et al., Proc. Natl. Acad. Sci. USA 99:16105-16110 (2002); Blyszczuk et al., Proc. Natl. Acad. Sci. USA 100:998-1003 (2003)). Insulin-producing clusters can also be obtained from human ES cells (Segev et al., Stem Cells 22:265-274 (2004)). These clusters express insulin, glucagon, and somatostatin. Several groups have reported the successful isolation and differentiation of stem cells derived from adult pancreatic ductal structures expressing endocrine hormones (Peck et al., Diabetes 44:10A (1995); Cornelius, Horm. Metab Res. 29:271-277 (1997); Ramiya et al., Nat. Med. 6:278-282 (2000); Bonner-Weir et al., Proc. Natl. Acad. Sci. USA 97:7999-8004 (2000); Rooman, Diabetologia 43:907-914 (2000); Gmyr et al., Cell Transplant. 10:109-121 (2001)). Currently, duct, acinar, and islet cells may contain cell populations capable of differentiation, trans-differentiation (differentiation along a pathway not normally followed), or de-differentiation into cells that have the potential to become endocrine cells (Peck et al., Transpl. Immunol. 12:259-272 (2004)). These adult stem cells can be cultured for the enrichment of multi-cell, islet-like structures which are then further matured in vivo (Peck et al., Transpl. Immunol. 12:259-272 (2004)). These islet-like structures can reverse the diabetic state in NOD mice within a week (Ramiya et al., Nat. Med. 6:278-282 (2000)) and in these NOD mice, there was no incidence of autoimmune recurrence.

Xenotransplantation. Xenotransplantation, or transplantation of tissue from one species to another, for example from animal to human, offers a potential solution to the tissue supply problem encountered in islet transplantation. Porcine and bovine islets as well as fish Brockman bodies are all potential sources of tissue for human islet transplantation (Korbutt et al., Annals New York Academy of Sciences 831:294-303 (1997); Marchetti et al., Diabetes 44:375-381 (1995); Wright et al., Cell Transplant. 10:125-143 (2001)). Using pigs, for example, as a source of tissue for islet transplantation offers the advantages of being inexpensive, readily available, ethically acceptable, can be housed in pathogen-free environments, and their islets exhibit morphological and physiological characteristics similar to human islets (Binette et al., Ann. N. Y. Acad. Sci. 944:47-61 (2001)). Porcine insulin is also structurally similar to human insulin and has been used for the treatment of type 1 diabetes for decades. Alternatively, transgenic pigs expressing human insulin are also useful in the methods of the invention. Additionally, neonatal porcine islets are the best candidate for eventual transplantation into humans (Korbutt et al., Ann. N. Y. Acad. Sci. 831:294-303 (1997)), as adult porcine islets are fragile and difficult to maintain in tissue culture and exhibit poor insulin secretory response to glucose (Ricordi et al., Surgery 107:688-694 (1990); van Deijnen et al., Cell Tissue Res. 267:139-146 (1992); Korsgren et al., Diabetologia 34:379-386 (1991)). Neonatal porcine islets, however, can be isolated in large numbers, show a potential for growth in vitro and in vivo, show excellent ability to respond to glucose challenge and are capable of restoring euglycemia in diabetic mice (Korbutt et al., J. Clin. Invest 97:2119-2129 (1996)).

Finally, the use of fish Brockmann bodies holds an advantage over neonatal porcine islets in that they do not require a lengthy isolation procedure and can be easily microdissected (Yang et al., Cell Transplant. 4:621-628 (1995)). Fish Brockmann bodies, like neonatal porcine islets, are subject to hyperacute rejection. Through the use of a granzyme B inhibitory serpin, this hyper acute rejection may be overcome. Microencapsulation of fish Brockmann bodies has been shown to be possible, and encapsulated Brockmann bodies are capable of restoring euglycemia in diabetic mice (Yang et al., Transplantation 64:28-32 (1997)). Additionally, no endogenous retrovirus in fish Brockmann bodies has been identified that can potentially be transmitted to the human population.

As the issue of hyperacute rejection of xenografts in humans has presented a major obstacle to widespread clinical applicability, xeno-transplantation of cells transgeneically modified to produce a granzyme B inhibitory serpin (e.g., serpina3n) or transplantation of two cell types, one of which expresses a granzyme B inhibitory serpin, may be used to overcome this obstacle.

Combination Therapy

Any method of the invention, for example, a treatment method or a transplantation method, may be performed in conjunction with an additional therapy (e.g., an immunosuppressive therapy) as is known in the art. Examples of immunosuppressive agents that may be used in combination therapy include cylcosporine, prednisone, azathioprine, tacrolimus (FK506), mycophenolate moefetil, sirolimus, OKT3, ATGAM, thymoglobulin, and monoclonal antibodies. In one embodiment, the patient in need of immunosuppressive therapy has autoimmune disease such as rheumatoid arthritis, treatment and transplantation methods of the invention may be combined with a treatment known in the art (e.g., methotrexate, Etanercept, Remicade).

The following examples are intended to illustration rather than limit the invention.

### EXAMPLE 1

### Xenotransplantation of a Porcine Heart into a Primate

Granzyme B inhibitory activity (e.g., activity of serpina3n) may be used to overcome immune rejection of a transplanted organ. Xenotransplantation, for example, using organs transplanted from a pig can provide a readily available source of organs such as heart; however, immune rejection of organs presents a major obstacle to widespread clinical adoption. By using the methods of the present invention, this obstacle may be overcome. To this end, a transgenic pig engineered to express a granzyme B inhibitory serpin (e.g., serpina3n) may be generated using methods known in the art, e.g., as described in Velander et al. (Proc. Natl. Acad. Sci. USA 89, 12003-12007 (1992)). The transgene includes a promoter operably linked to a gene encoding a granzyme B inhibitory serpin such as serpina3n, where the promoter is capable of driving expression in cardiac tissue of the heart.

Transplantation of a pig heart (e.g., from a serpina3n transgenic pig) into a patient, for example, a primate such as a baboon, is described by Schmoeckel et al. (Transplantation 65:1570-1577 (1998)).

The transplanted heart which expresses the granzyme B inhibitory serpin (e.g., serpina3n) at a level sufficient to reduce an immune response in the patient may therefore avoid immune rejection, as the production of the serpin e.g., serpina3n) will decrease the cell-killing activity of the CTL cells near the transplanted heart. Unlike administration of systemic immunosuppressive therapies which decrease immune response in all tissues and organs, the transplanted heart will reduce immune response locally, where it is required. This may result in fewer side effects as compared to patient receiving systemic therapies (e.g., greater susceptibility to infections).

### EXAMPLE 2

### Transplantation of Porcine Islet Cells Expressing Serpina3n

Porcine islet cells may be especially useful in transplantation for treatment of diabetes. As noted above, neonatal porcine islets are the best candidate for eventual transplantation into humans (Korbutt et al., Annals New York Academy of Sciences 831:294-303 (1997)), as compared to adult porcine islets, which are fragile and difficult to maintain in tissue culture, and fetal porcine islets, which exhibit poor insulin secretory response to glucose (Ricordi et al., Surgery 107:688-694 (1990); van Deijnen et al., Cell Tissue Res. 267:139-146 (1992); Korsgren et al., Diabetologia 34:379-386 (1991)).

Isolation and growth of neonatal porcine islets may be carried out as described by Korbutt et al. (J. Clin. Invest 97:2119-2129 (1996)). Cells prepared in this manner may be either derived from a transgenic pig expressing a gene encoding a granzyme B inhibitory serpin such as serpina3n, or cells from a wild-type pig may be transfected following isolation (e.g., using transfection techniques standard in the art such as a retroviral vector) to generate cells that express a granzyme B inhibitory serpin such as serpina3n. The cells can then be transplanted as described in Korbutt et al., supra. Transplantation of pig islets into primates is known in the art and described by Komoda et al. (Xenotransplantation 12:209-216 (2005)). Typically, cells are transplanted in the liver, pancreas, or omental pouch of a patient. The use of serpina3n-expressing islet cells may reduce or eliminate the need for exogenous or systemic immunosuppressive treatments to ensure that the transplanted cells are not rejected by the host.

### EXAMPLE 3

### Transplantation of Fish Islet Cells Expressing Serpina3n

The use of fish Brockmann bodies in transplantation to treat diabetes are advantageous in that they can be isolated without a lengthy procedure; also, no endogenous retrovirus transmittable to humans has been identified in fish. Microencapsulation of fish Brockmann bodies is possible, and encapsulated Brockmann bodies can restore euglycemia in diabetic mice. However, wild-type fish Brockmann bodies are subject to hyperacute immune rejection in humans, and the endogenous insulin in Brockmann bodies is less suitable than human or porcine insulin for treatment of diabetes in humans. As in the above example, the methods of the present invention may be used to overcome these limitations in treatment of patients in need of insulin such. To this end, transgenic fish expressing two exogenous genes, (1) a gene encoding a granzyme B inhibitory serpin (e.g., serpina3n) and (2) a gene encoding human insulin may be generated. A promoter operably linked to each of these two genes capable of driving expression in Brockmann bodies is selected.

Brockman bodies from an above-described transgenic fish can be microdissected, as described in Yang et al. (Cell Transplant. 4:621-628 (1995)). These cell can then transplanted into a patient (e.g., into the liver or pancreas of a mammal). The transplanted cells express a granzyme B inhibitory serpin (e.g., serpina3n), thereby reducing the immune response against the cells, which, in turn, may prevent immune rejection of the transplanted cells. The reduction of immune response is localized to the area of the transplanted cells, thereby reducing the likelihood of undesirable side effects.

### Other Embodiments

All patents, patent applications, and publications mentioned in this specification are herein incorporated by reference to the same extent as if each independent patent, patent application, or publication was specifically and individually indicated to be incorporated by reference.

From the foregoing description, it is apparent that variations and modifications may be made to the invention described herein to adopt it to various usages and conditions. Such embodiments are also within the scope of the following claims.

What is claimed is:

1. A method for treating a patient in need of immunosuppression, said method comprising administering to said patient a therapeutically effective amount of a composition comprising a granzyme B inhibitory serpin or a granzyme B inhibitory fragment thereof in an amount sufficient to decrease an immune response of said patient.

2. The method of claim 1, wherein said serpin is serpina3n or a modified human α1-antichymotrypsin.

3. The method of claim 1, wherein said patient has an autoimmune disorder, an inflammatory vascular disease, or an inflammatory neuronal disease.

4. The method of claim 3, wherein said autoimmune disorder is diabetes or rheumatoid arthritis.

5. The method of claim 1, wherein said immune response is mediated by cytotoxic T lymphocytes.

6. The method of claim 1, wherein said patient is the recipient of a transplanted cell.

7. The method of claim 6, wherein said cell is a cell in a transplanted organ.

8. The method of claim 7, wherein said organ is a heart, liver, kidney, pancreas, or lung.

9. A method for transplanting a cell into a mammal, said method comprising:
(a) providing a composition comprising a first cell comprising a first heterologous polynucleotide encoding a granzyme B inhibitory serpin or a granzyme B inhibitory fragment thereof, wherein said cell is a eukaryotic cell; and
(b) introducing said composition into said mammal.

10. The method of claim 9, wherein said serpin is serpina3n or a modified human α1-antichymotrypsin.

11. The method of claim 9, wherein said mammal is a human.

12. The method of claim 9, wherein said first cell is an islet cell, human cell, stem cell, porcine cell, or fish cell.

13. The method of claim 12, wherein said fish cell is a Brockmann body.

14. The method of claim 9, wherein said composition further comprises a second cell.

15. The method of claim 14, wherein said second cell is an islet cell.

16. The method of claim 9, wherein said cell is a cell in a transplanted organ.

17. The method of claim 16, wherein said organ is a heart, liver, kidney, pancreas, or lung.

18. The method of claim 9, wherein said cell further comprises a second heterologous polynucleotide encoding a second polypeptide.

19. The method of claim 18, wherein said second polypeptide is insulin.

20. A composition comprising a cell comprising a heterologous polynucleotide sequence encoding a granzyme B inhibitory serpin or a granzyme B inhibitory fragment thereof, wherein said cell is a eukaryotic cell.

21. The composition of claim 20, wherein said serpin is serpina3n or a modified human α1-antichymotrypsin.

22. The composition of claim 20, wherein said polynucleotide sequence is operably linked to a promoter.

23. The composition of claim 20, wherein said cell is a mammalian cell, islet cell, or fish cell.

24. The composition of claim 23, wherein said mammalian cell is a human cell or a porcine cell.

25. The composition of claim 20, further comprising a second cell for transplantation.

26. The composition of claim 25, wherein said second cell is an islet cell.

27. A pharmaceutical composition comprising a granzyme B inhibitory serpin or a granzyme B inhibitory fragment thereof and a pharmaceutically acceptable carrier.

28. The pharmaceutical composition of claim 27, wherein said serpin is serpina3n or a modified human α1-antichymotrypsin.

29. The pharmaceutical composition of claim 27, wherein said carrier is suitable for parenteral or intravenous administration.

30. A pharmaceutical composition comprising a polynucleotide encoding a granzyme B inhibitory serpin or a granzyme B inhibitory fragment thereof and a pharmaceutically acceptable carrier.

31. The pharmaceutical composition of claim 30, wherein said serpin is serpina3n or a modified human α1-antichymotrypsin.

32. A composition comprising a vector comprising a polynucleotide encoding a granzyme B inhibitory serpin or a granzyme B inhibitory fragment thereof.

33. The composition of claim 32, wherein said vector is a viral vector.

34. A transgenic, non-human animal comprising a first heterologous polynucleotide encoding a granzyme B inhibitory serpin or granzyme B inhibitory fragment thereof, wherein said serpin or said fragment is operably linked to a promoter capable of expressing said polynucleotide in at least one tissue of said transgenic animal.

35. The transgenic animal of claim 34, wherein said animal is a pig or a fish.

36. The transgenic animal of claim 34, wherein said transgenic animal further comprises a second heterologous polynucleotide.

37. The transgenic animal of claim 36, wherein said second polynucleotide encodes human insulin.

38. The transgenic animal of claim 34, wherein said tissue is cardiac tissue or pancreatic tissue.

39. A method for transplanting tissue from a transgenic animal into a patient, said method comprising:
(a) providing a composition comprising a tissue from the transgenic animal of claim 34; and
(b) introducing said composition into said patient.

40. The method of claim 39, wherein said transgenic animal is a pig.

41. The method of claim 39, wherein said tissue comprises a heart, liver, kidney, pancreas, or lung.

42. The method of claim 39, wherein said tissue comprises an islet cell.

43. The method of claim 39, wherein said patient is a human.
